(19) United States
(12) Patent Application Publication
(10) Pub. No.: US 2026/0123838 A1
(43) Pub. Date: May 7, 2026

(54) WEARABLE DEVICE FOR NONINVASIVE BODY TEMPERATURE MEASUREMENT

(71) Applicant: Masimo Corporation, Irvine, CA (US)

(72) Inventors: Ammar Al-Ali, San Juan Capistrano, CA (US); Stephen Scruggs, Newport Beach, CA (US); Joel Amposta, Irvine, CA (US); Valery G. Telfort, Irvine, CA (US)

(21) Appl. No.: 19/420,511

(22) Filed: Dec. 15, 2025

Related U.S. Application Data
(63) Continuation of application No. 17/933,604, filed on Sep. 20, 2022, now Pat. No. 12,521,021.
(60) Provisional application No. 63/261,500, filed on Sep. 22, 2021.

(51) Int. Cl. A61B 5/01; A61B 5/00; G01K 1/16; G01K 7/22; G01K 13/20

(52) U.S. Cl. CPC A61B 5/01; A61B 5/6832; G01K 1/16; G01K 7/22; G01K 13/20; A61B 2562/0271; A61B 2562/043

(57) ABSTRACT

A wearable device configured to secure to skin of a user and noninvasively measure body temperature of the user can include first and second pairs of temperature sensors configured to generate one or more signals responsive to detected thermal energy, a thermally conductive element positioned at least partially between the second pair of temperature sensors, and one or more hardware processors configured to receive the one or more signals generated by each of said first and second pairs of temperature sensors and determine one or more body temperature values of the user based on at least comparisons between different ones of the first and second pairs of temperature sensors. In some implementations, the wearable device includes thermally conductive probes for transmitting thermal energy toward ones of the first and second pairs of temperature sensors and a substrate positioned between the probes and the skin.

### INCORPORATION BY REFERENCE TO ANY PRIORITY APPLICATIONS

The present application is a continuation of U.S. application Ser. No. 17/933,604, filed Sep. 20, 2022, titled “Wearable Device for Noninvasive Body Temperature Measurement”, which claims priority to U.S. Provisional Application No. 63/261,500, filed Sep. 22, 2021, titled “Wearable Device for Noninvasive Body Temperature Measurement”, which is hereby incorporated by reference in its entirety.

### TECHNICAL FIELD

The present disclosure relates to devices, methods, and/or systems for monitoring a subject's physiological information. More specifically, the present disclosure describes, among other things, a wearable device for measuring a subject's body temperature.

### BACKGROUND

Core body temperature is an important vital sign used by clinicians to monitor and/or manage the condition of a subject (for example, a patient). Core body temperature is the internal temperature of a subject. Internal body temperatures are typically maintained within a specific range in order for the body to carry out essential functions. Variations in core body temperature can be indicative of a deteriorating condition of a subject and can negatively impact the body's ability to maintain critical life-sustaining functions.

### SUMMARY

Despite the importance of core body temperature as a vital sign, many commonly employed devices, methods, and/or systems for estimating (via noninvasive or minimally invasive means) core body temperature based on skin surface or peripheral measurements lack accuracy. Skin surface temperature, typically measured using single point measurement devices or heat flux measurement devices, can vary dramatically from core body temperature in some cases depending on physiology of the subject such as properties of the subject's skin (for example, thickness, impedance), condition of the subject's skin when measurements are taken (for example, moisture/sweat), environment of the subject, perfusion, and/or other conditions. Temperature measurements obtained with a thermometer at a subject's periphery (such as at the subject's armpit, rectum, or under a subject's tongue) also do not represent a true measurement of internal body temperature, but rather, simply an approximation. The present disclosure provides improved devices, methods, and systems for noninvasively determining a subject's internal body temperature based upon temperature measurements obtained from the subject's skin.

Various implementations of the disclosed wearable devices include multiple temperature sensors operably positioned in different locations with respect to one another and with respect to the wearer's skin when in use. Such configurations can allow temperature to be determined at each of these different locations and compared with one another. In some implementations, thermal paths (which may be referred to as “thermal flow paths” or “heat flow paths” or “thermal energy paths”) between pairs of temperature sensors are defined by air and/or a thermally conductive element, which can provide additional information where thermal properties (for example, thermal conductivity values) are known. Temperature values at various ones of the temperature sensors and differences between such values can be utilized to provide more accurate estimates of internal body temperature of a subject. Some implementations of the wearable devices disclosed herein include two pairs of temperature sensors aligned with one another, where one of each pair is positioned farther from the subject's skin (when the wearable device is in use) and the other one of each pair is positioned closer to the subject's skin. Some implementations include an air gap (which can act as a thermal insulator) between one of such pairs and a thermally conductive element (for example, a metallic material) between the other one of such pairs. Temperature values determined based on each of the temperature sensors (and/or each of the pairs of temperature sensors) can be compared and/or otherwise utilized to approximate internal body temperature value(s) of the subject. In various implementations, one or more thermally conductive probes can be utilized to transmit energy from a substrate of the wearable device (which can adhere to the subject's skin) toward aligned temperature sensor(s).

Some implementations of the disclosed wearable devices (or portions of such devices) can be disposable, which can reduce the risk of cross-contamination between multiple subjects. Some implementations of the disclosed wearable devices (or portions of such devices) can be waterproof, thereby allowing the subject to carry out ordinary activities (for example, showering) without disrupting operation of the wearable device. Some implementations of the disclosed wearable devices include two separable components (which may also be referred to as “separate portions” or “first and second portions”). In such implementations, a first one of the components can be configured to secure to a portion of a subject (for example, skin of the user) and a second one of the components can be configured to secure (for example, removably secure) to the first component. In some implementations, the first and second components are configured such that separation thereof is inhibited or prevented when the first component is secured to the subject but is allowed when the first component is not secured to the subject. Such implementations can be advantageous in scenarios where it is desirable to inhibit or prevent a user from interfering with operation of the wearable device. For example, in some implementations, the wearable device includes a button configured to transition the wearable device (or a portion thereof such as the second component discussed above) between non-operational and operational modes. In some of such implementations, such button is inaccessible (for example, to the subject wearing the wearable device and/or to another person, such as a caregiver) unless the first and second components are separated from one another. Such implementations can advantageously prevent a subject (for example, a child) from intentionally or unintentionally turning the wearable device off when the wearable device is secured to the subject (which can ensure proper compliance in some situations).

Disclosed herein is a wearable device configured to secure to skin of a user and noninvasively measure body temperature of the user, the wearable device comprising: a first pair of temperature sensors, said first pair of temperature sensors comprising a first temperature sensor and a second temperature sensor, each of said first and second temperature sensors configured to generate one or more signals responsive to detected thermal energy, said first temperature sensor operably positioned to be closer to the user's skin than the second temperature sensor when the wearable device is secured to the user's skin; a second pair of temperature sensors spaced from said first pair of temperature sensors, said second pair of temperature sensors comprising a third temperature sensor and a fourth temperature sensor, each of said third and fourth temperature sensors configured to generate one or more signals responsive to detected thermal energy, said third temperature sensor operably positioned to be closer to the user's skin than the fourth temperature sensor when the wearable device is secured to the user's skin; a thermally conductive element positioned at least partially between the third and fourth temperature sensors; and one or more hardware processors. The one or more hardware processors can be configured to: receive said one or more signals generated by each of said first and second temperature sensors; and receive said one or more signals generated by each of said third and fourth temperature sensors. The one or more hardware processors can be configured to determine one or more body temperature values of the user based on at least: a first comparison between said one or more signals generated by said first temperature sensor and said one or more signals generated by said second temperature sensor; and a second comparison between said one or more signals generated by said third temperature sensor and said one or more signals generated by said fourth temperature sensor.

In some implementations, the first pair of temperature sensors are thermally insulated from one another by an air gap. In some implementations: the wearable device further comprises a first circuit board and a second circuit board, the first and second circuit boards spaced from one another, the first circuit board positioned closer to the skin of the user than the second circuit board when the wearable device is secured to the user's skin; said first temperature sensor is mounted to the first circuit board and the second temperature sensor is mounted to the second circuit board; said third temperature sensor is mounted to the first circuit board and spaced from the first temperature sensor; said fourth temperature sensor is mounted to the second circuit board and spaced from the second temperature sensor; a distance between the first temperature sensor and the second circuit board at least partially defines said air gap; and said thermally conductive element is positioned between the third temperature sensor and a portion of the second circuit board that is adjacent to the fourth temperature sensor.

In some implementations: said first circuit board comprises a first surface and a second surface; said second circuit board comprises a first surface and a second surface; said first surface of the first circuit board faces toward the second surface of the second circuit board; said first and third temperature sensors are mounted on the first surface of the first circuit board; said second and fourth temperature sensors are mounted on the first surface of the second circuit board; and said thermally conductive element is positioned between the third temperature sensor and a portion of the second surface of the second circuit board that is adjacent to the fourth temperature sensor. In some implementations, said second circuit board comprises at least one opening positioned between the fourth temperature sensor and a portion of said thermally conductive element, said at least one opening configured to allow thermal energy to pass from the thermally conductive element through the second circuit board and to the fourth temperature sensor. In some implementations, said first and second temperature sensors are substantially aligned with one another and wherein said third and fourth temperature sensors are substantially aligned with one another.

In some implementations, said thermally conductive element comprises a metal strip. In some implementations, said metal strip comprises copper. In some implementations, the first and second circuit boards are arranged to be substantially parallel to one another. In some implementations, said one or more hardware processors are further configured to determine said one or more body temperature values based on: a third comparison between said one or more signals generated by at least one of the first pair of temperature sensors and said one or more signals generated by at least one of the second pair of temperature sensors. In some implementations, said one or more hardware processors are further configured to determine said one or more body temperature values based on: a third comparison between said one or more signals generated by the first temperature sensor and said one or more signals generated by the fourth temperature sensor.

In some implementations, the wearable device comprises a first portion configured to be secured to the user's skin and a second portion configured to removably secure to the first portion, and wherein the one or more hardware processors, the thermally conductive element, the first pair of temperature sensors, and the second pair of temperature sensors are positioned within the second portion of the wearable device. In some implementations, the first portion comprises a frame and a substrate coupled to the frame, the substrate configured to secure to the user's skin, and wherein the second portion comprises a housing. In some implementations, the housing comprises a first shell and a second shell, and wherein the first and second shells are permanently secured together. In some implementations, none of the first temperature sensor, second temperature sensor, third temperature sensor, and fourth temperature sensor contact the user's skin when the wearable device is secured to the user.

Disclosed herein is a wearable device configured to secure to skin of a user and noninvasively measure body temperature of the user, the wearable device comprising: a first pair of temperature sensors, said first pair of temperature sensors comprising a first temperature sensor and a second temperature sensor, each of said first and second temperature sensors configured to generate one or more signals responsive to detected thermal energy, said first temperature sensor operably positioned to be closer to the user's skin than the second temperature sensor when the wearable device is secured to the user's skin; a second pair of temperature sensors spaced from said first pair of temperature sensors, said second pair of temperature sensors comprising a third temperature sensor and a fourth temperature sensor, each of said third and fourth temperature sensors configured to generate one or more signals responsive to detected thermal energy, said third temperature sensor operably positioned to be closer to the user's skin than the fourth temperature sensor when the wearable device is secured to the user's skin; a thermally conductive element positioned at least partially between the third and fourth temperature sensors; and one or more hardware processors. The one or more hardware processors can be configured to: receive said one or more signals generated by each of said first and second temperature sensors; receive said one or more signals generated by each of said third and fourth temperature sensors; determine a first temperature gradient based on said one or more signals generated by each of the first pair of temperature sensors; determine a second temperature gradient based on said one or more signals generated by each of the second pair of temperature sensors; and determine one or more body temperature values of the user based on at least said first and second temperature gradients.

In some implementations, the first pair of temperature sensors are thermally insulated from one another by an air gap. In some implementations: the wearable device further comprises a first circuit board and a second circuit board, the first and second circuit boards spaced from one another, the first circuit board positioned closer to the skin of the user than the second circuit board when the wearable device is secured to the user's skin; said first temperature sensor is mounted to the first circuit board and said second temperature sensor is mounted to the second circuit board; said third temperature sensor is mounted to the first circuit board and spaced from the first temperature sensor; said fourth temperature sensor is mounted to the second circuit board and spaced from the second temperature sensor; a distance between the first temperature sensor and the second circuit board at least partially defines said air gap; and said thermally conductive element is positioned between the third temperature sensor and a portion of the second circuit board that is adjacent to the fourth temperature sensor. In some implementations: said first circuit board comprises a first surface and a second surface; said second circuit board comprises a first surface and a second surface; said first surface of the first circuit board faces toward the second surface of the second circuit board; said first and third temperature sensors are mounted on the first surface of the first circuit board; said second and fourth temperature sensors are mounted on the first surface of the second circuit board; and said thermally conductive element is positioned between the third temperature sensor and a portion of the second surface of the second circuit board that is adjacent to the fourth temperature sensor. In some implementations, said second circuit board comprises at least one opening positioned between the fourth temperature sensor and a portion of said thermally conductive element, said at least one opening configured to allow thermal energy to pass from the thermally conductive element through the second circuit board and to the fourth temperature sensor.

In some implementations, said first and second temperature sensors are substantially aligned with one another and said third and fourth temperature sensors are substantially aligned with one another. In some implementations, said thermally conductive element comprises a metal strip. In some implementations, said metal strip comprises copper. In some implementations, the first and second circuit boards are arranged to be substantially parallel to one another. In some implementations, the wearable device comprises a first portion configured to be secured to the user's skin and a second portion configured to removably secure to the first portion, and the one or more hardware processors, the thermally conductive element, the first pair of temperature sensors, and the second pair of temperature sensors are positioned within the second portion of the wearable device. In some implementations, the first portion comprises a frame and a substrate coupled to the frame, the substrate configured to secure to the user's skin, and the second portion comprises a housing. In some implementations, the housing comprises a first shell and a second shell, and the first and second shells are permanently secured together.

Disclosed herein is a wearable device configured for noninvasive measurement of body temperature of a user, the wearable device comprising: a housing; a substrate coupled to the housing and configured to secure to skin of the user; a circuit board positioned within a portion of the housing and comprising a first surface and a second surface, wherein the second surface is configured to be positioned closer to the substrate than the first surface when the wearable device is in use; a first temperature sensor mounted to the first surface of the circuit board; a first thermally conductive probe positioned adjacent the second surface of the circuit board and substantially aligned with the first temperature sensor; a second temperature sensor mounted to the first surface of the circuit board and spaced away from the first temperature sensor; and a second thermally conductive probe positioned adjacent the second surface of the circuit board and substantially aligned with the second temperature sensor, the second thermally conductive probe spaced away from the first thermally conductive probe. In some implementations: the first and second thermally conductive probes contact the substrate and the substrate is positioned between the user's skin and the first and second thermally conductive probes when the wearable device is in use; the first and second thermally conductive probes are configured to transmit thermal energy of the user toward the first and second temperature sensors; and the wearable device is configured to determine body temperature of the user based at least on one or more signals generated by each of the first and second temperature sensors responsive to said thermal energy.

In some implementations, the first thermally conductive probe comprises a first end and a second end opposite the first end, the first end configured to contact the substrate when the wearable device is in use and the second end in contact with the second surface of the circuit board. In some implementations, the second thermally conductive probe comprises a first end and a second end opposite the first end, the first end configured to contact the substrate when the wearable device is in use and the second end in contact with the second surface of the circuit board. In some implementations, the wearable device further comprises: at least one opening in the circuit board positioned at least partially between the second end of the first thermally conductive probe and the first temperature sensor, said at least one opening configured to allow thermal energy to pass from the second end of the first thermally conductive probe through the circuit board and to the first temperature sensor; and at least one opening in the circuit board positioned at least partially between the second end of the second thermally conductive probe and the second temperature sensor, said at least one opening configured to allow thermal energy to pass from the second end of the second thermally conductive probe through the circuit board and to the second temperature sensor.

In some implementations: said at least one opening in the circuit board positioned at least partially between the second end of the first thermally conductive probe and the first temperature sensor comprises a first plurality of openings; and said at least one opening in the circuit board positioned at least partially between the second end of the second thermally conductive probe and the second temperature sensor comprises a second plurality of openings.

In some implementations, each of said first plurality of openings is filled with a thermally conductive material, and wherein each of said second plurality of openings is filled with said thermally conductive material. In some implementations, said thermally conductive material comprises copper. In some implementations, each of said first plurality of openings is not filled with a material, and wherein each of said second plurality of opening is not filled with a material. In some implementations, said first temperature sensor and said first thermally conductive probe are substantially aligned along a first axis and wherein said second temperature sensor and second thermally conductive probe are substantially aligned along a second axis, and wherein the first and second axes are substantially parallel to one another. In some implementations, each of the first and second thermally conductive probes comprise a metallic material. In some implementations, each of the first and second temperature sensors is an integrated circuit (IC) temperature sensor. In some implementations, each of the first and second temperature sensors is a thermistor. In some implementations, a cross-section of each of the first and second thermally conductive probes is circular.

In some implementations, said wearable device comprises: a first portion comprising said substrate and a frame coupled to said substrate; and a second portion comprising said housing that is removably securable to said frame, wherein said circuit board and said first and second temperature sensors are positioned within said housing. In some implementations, said housing comprises a first opening and a second opening, and wherein the first thermally conductive probe extends through said first opening and said second thermally conductive probe extends through said second opening. In some implementations: the housing comprises a first shell and a second shell; the first shell is configured to be positioned closer to the substrate than the second shell when the housing is secured to the frame; and the first shell comprises the first and second openings. In some implementations, the wearable device further comprises one or more hardware processors configured to determine said body temperature of the user based at least on said one or more signals generated by each of the first and second temperature sensors responsive to said thermal energy.

Disclosed herein is a wearable device configured for noninvasive measurement of body temperature of a user, the wearable device comprising a first portion and a second portion configured to be removably secured to the first portion of the wearable device. The first portion can comprise: a frame; and a substrate coupled to the frame and configured to secure to skin of the user. The second portion can comprise: a housing configured to mechanically connect to the frame of the first portion of the wearable device; and a first temperature sensor positioned within the housing. The wearable device can be configured to determine body temperature based at least on one or more signals generated by the first temperature sensor responsive to thermal energy.

In some implementations, the first portion of the wearable device does not include any electronic components. In some implementations, the first portion of the wearable device does not include a temperature sensor. In some implementations, the first portion of the wearable device does not include a power source. In some implementations, the second portion of the wearable device further comprises a second temperature sensor positioned within the housing and spaced from the first temperature sensor, wherein the wearable device is configured to determine body temperature based at least on one or more signals generated by the first and second temperature sensors responsive to said thermal energy. In some implementations: the second portion of the wearable device further comprises a circuit board positioned within the housing and comprising a first surface and a second surface; the second surface is positioned closer to the substrate than the first surface when the first and second portions of the wearable device are secured together; and the first and second temperature sensors are mounted to the first surface of the circuit board.

In some implementations: the frame comprises an opening; the substrate is configured to be positioned between the opening of the frame and the user's skin when the wearable device is in use; the housing comprises a first opening. The second portion of the wearable device can further comprise: a first thermally conductive probe positioned adjacent the second surface of the circuit board and substantially aligned with the first temperature sensor, the first thermally conductive probe extending through the first opening of the housing. The opening of the frame can allow the first thermally conductive probe to contact the substrate when the first and second portions of the wearable device are secured together. The first thermally conductive probe can transmit thermal energy of the user toward the first temperature sensor. In some implementations, the housing further comprises a second opening that is spaced from the first opening of the housing and the second portion of the wearable device further comprises: a second thermally conductive probe positioned adjacent the second surface of the circuit board and substantially aligned with the second temperature sensor, the second thermally conductive probe spaced away from the first thermally conductive probe and extending through the second opening of the housing. The opening of the frame can allow the second thermally conductive probe to contact the substrate when the first and second portions of the wearable device are secured together. The second thermally conductive probe can transmit thermal energy of the user toward the second temperature sensor.

In some implementations, the housing comprises a first shell and a second shell, the first shell positioned closer to the substrate than the second shell when the first and second portions of the wearable device are secured together. In some implementations, the housing comprises at least one recess and the frame comprises at least one protrusion configured to engage the at least one recess to allow the first portion to secure to the second portion. In some implementations: the frame comprises a rim, a first arm extending outward from the rim, and a second arm extending outward from the rim and spaced from the first arm, said rim comprising said opening of the frame; the first arm comprises a first protrusion and the second arm comprises a second protrusion; the housing comprises a first recess and a second recess spaced from the first recess; and the first and second protrusions are configured to engage the first and second recesses to allow the first portion to secure to the second portion. In some implementations: the first arm is located at a first end of the frame and the second arm is located at a second end of the frame that is opposite the first end of the frame; and the first recess is located at a first end of the housing and the second recess is located at a second end of the housing that is opposite the first end of the housing.

In some implementations: the frame further comprises a first side and a second side opposite the first side; and application of opposing forces on the first and second sides of the frame causes movement of the first and second protrusions out of engagement with the first and second recesses of the housing such that the housing can be removed from the frame. In some implementations, said first and second sides of the frame are defined by the rim of the frame. In some implementations: said application of opposing forces on the first and second sides of the frame causes said first and second arms to flex outward from each other in opposite directions. In some implementations: said application of said opposing forces on the first and second sides of the frame causes the first and second arms to move from a first position to a second position; free ends of the first and second arms are positioned closer to one another when in the first position than when in the second position; and removal of said application of said opposing forces on the first and second sides of the frame causes the first and second arms to move from the second position to the first position. In some implementations, the first and second arms extend generally perpendicular to the rim. In some implementations, the first and second arms extend in the same direction. In some implementations, the second portion of the wearable device further comprises a button configured to allow the wearable device to be transitioned from an operational mode to a non-operational mode, and wherein, said button faces toward the substrate when the first and second portions are secured together.

In some implementations, the second portion of the wearable device further comprises a button configured to allow the wearable device to be transitioned from an operational mode to a non-operational mode, and wherein, said button is inaccessible when the first and second portions are secured together. In some implementations, when the wearable device is in said non-operational mode, the wearable device does not determine body temperature of the user. In some implementations, the second portion of the wearable device comprises a communication module configured to allow the wearable device to wirelessly transmit said determined body temperature to a separate device.

Disclosed herein is a wearable device configured for noninvasive measurement of body temperature of a user, the wearable device comprising: a housing; a substrate coupled to the housing and configured to secure to skin of the user; a first circuit board positioned within a portion of the housing and comprising a first surface and a second surface, wherein the second surface of the first circuit board is configured to be positioned closer to the substrate than the first surface of the first circuit board; a second circuit board positioned within a portion of the housing and spaced from the first circuit board by a gap, the second circuit board comprising a first surface and a second surface, wherein the second circuit board is configured to be positioned farther from the substrate than the first circuit board; a first temperature sensor mounted to the first surface of the first circuit board; a thermally conductive probe positioned adjacent the second surface of the first circuit board and substantially aligned with the first temperature sensor, wherein the thermally conductive probe contacts the substrate and the substrate is positioned between the user's skin and the thermally conductive probe when the wearable device is in use, and wherein the thermally conductive probe is configured to transmit thermal energy of the user toward the first temperature sensor; and a second temperature sensor mounted to the first surface of the second circuit board and substantially aligned with the first temperature sensor. The wearable device can be configured to determine body temperature based at least on one or more signals generated by the first and second temperature sensors.

In some implementations, the first surface of the first circuit board faces toward the second surface of the second circuit board. In some implementations, the second surface of the second circuit board is configured to be positioned closer to the substrate than the first surface of the second circuit board. In some implementations, the wearable device further comprises a thermally conductive element having a first end secured to the first temperature sensor and a second end secured to a portion of the second surface of the second circuit board that is substantially aligned with the second temperature sensor, wherein the thermally conductive element is configured to at least partially transmit said thermal energy from the first temperature sensor to the second temperature sensor. In some implementations, said thermally conductive element comprises a metallic material. In some implementations, said thermally conductive element comprises copper. In some implementations, an air gap is positioned between the first temperature sensor and a portion of the second surface of the second circuit board that is substantially aligned with said second temperature sensor. In some implementations, the wearable device further comprises at least one hole in the first circuit board adjacent the first temperature sensor and at least one hole in the second circuit board adjacent the second temperature sensor. In some implementations, the wearable device further comprises a battery positioned between the first and second circuit boards. In some implementations, the wearable device further comprises: an antenna configured to allow the wearable device to wirelessly communicate with a separate device; and a support structure connected to the second circuit board and configured to position the antenna away from the first surface of the second circuit board, thereby positioning the antenna farther away from the battery and increasing a wireless range of the antenna.

Disclosed herein is a wearable device configured for noninvasive measurement of body temperature of a user, the wearable device comprising: a housing; a substrate configured to be removably coupled to the housing and configured to secure to skin of the user; a circuit board positioned within a portion of the housing and comprising a first surface and a second surface, wherein the second surface is configured to be positioned closer to the substrate than the first surface when the wearable device is in use; a first temperature sensor mounted to the first surface of the circuit board; a thermally conductive probe positioned adjacent the second surface of the circuit board and substantially aligned with the first temperature sensor, wherein the thermally conductive probe contacts the substrate and the substrate is positioned between the user's skin and the thermally conductive probe when the wearable device is in use, and wherein the thermally conductive probe is configured to transmit thermal energy of the user toward the first temperature sensor; a second temperature sensor mounted to the first surface of the circuit board and spaced away from the first temperature sensor; and a thermally conductive element connected to and extending between the first and second temperature sensors. The wearable device can be configured to determine body temperature based at least on one or more signals generated by the first and second temperature sensors.

Disclosed herein is a method of noninvasively measuring body temperature of a user with a wearable device, the method comprising: receiving one or more signals generated by each of a first pair of temperature sensors of the wearable device responsive to detected thermal energy, said first pair of temperature sensors comprising a first temperature sensor and a second temperature sensor, said first temperature sensor operably positioned to be closer to skin of the user than said second temperature sensor when the wearable device is secured to the user's skin; and receiving one or more signals generated by each of a second pair of temperature sensors of the wearable device responsive to detected thermal energy, said second pair of temperature sensors spaced from said first pair of temperature sensors and comprising a third temperature sensor and a fourth temperature sensor, said third temperature sensor operably positioned to be closer to skin of the user than said fourth temperature sensor when the wearable device is secured to the user's skin. The method can further comprise determining, with one or more hardware processors of the wearable device, one or more body temperature values of the user based on at least: a first comparison between the one or more signals generated by said first temperature sensor and the one or more signals generated by said second temperature sensor; and a second comparison between the one or more signals generated by said third temperature sensor and the one or more signals generated by said fourth temperature sensor.

In some implementations, the method further comprises allowing thermal energy to flow through a thermally conductive element positioned at least partially between the third and fourth temperature sensors. In some implementations, said thermally conductive element comprises copper. In some implementations, said determining said one or more body temperature values of the user is further based on a third comparison between said one or more signals generated by at least one of the first pair of temperature sensors and said one or more signals generated by at least one of the second pair of temperature sensors.

Disclosed herein is a method of noninvasively measuring body temperature of a user with a wearable device, the method comprising: receiving one or more signals generated by each of a first pair of temperature sensors of the wearable device responsive to detected thermal energy, said first pair of temperature sensors comprising a first temperature sensor and a second temperature sensor, said first temperature sensor operably positioned to be closer to skin of the user than said second temperature sensor when the wearable device is secured to the user's skin; and receiving one or more signals generated by each of a second pair of temperature sensors of the wearable device responsive to detected thermal energy, said second pair of temperature sensors spaced from said first pair of temperature sensors and comprising a third temperature sensor and a fourth temperature sensor, said third temperature sensor operably positioned to be closer to skin of the user than said fourth temperature sensor when the wearable device is secured to the user's skin. The method can further comprise determining with one or more hardware processors of the wearable device: a first temperature gradient based on the one or more signals generated by each of the first pair of temperature sensors; a second temperature gradient based on the one or more signals generated by each of the second pair of temperature sensors; and one or more body temperature values of the user based on at least said first and second temperature gradients.

Disclosed herein is a wearable device configured to secure to skin of a user, the wearable device comprising: a first pair of temperature sensors, said first pair of temperature sensors comprising a first temperature sensor and a second temperature sensor, said first temperature sensor operably positioned to be closer to the user's skin than the second temperature sensor when the wearable device is secured to the user's skin; and a second pair of temperature sensors spaced from said first pair of temperature sensors, said second pair of temperature sensors comprising a third temperature sensor and a fourth temperature sensor, said third temperature sensor operably positioned to be closer to the user's skin than the fourth temperature sensor when the wearable device is secured to the user's skin. In some implementations, the wearable device further comprises one or more hardware processors configured to: receive one or more signals generated by each of said first temperature sensor, second temperature sensor, third temperature sensor, and fourth temperature sensor responsive to thermal energy; and determine one or more body temperature values of the user based on at least: a first comparison between said one or more signals generated by said first temperature sensor and said one or more signals generated by said second temperature sensor; and a second comparison between said one or more signals generated by said third temperature sensor and said one or more signals generated by said fourth temperature sensor.

In some implementations, the wearable device further comprises a thermally conductive element positioned at least partially between the third and fourth temperature sensors. In some implementations, the first pair of temperature sensors are thermally insulated from one another by an air gap. In some implementations, the wearable device further comprises a first circuit board and a second circuit board, the first and second circuit boards spaced from one another, the first circuit board positioned closer to the skin of the user than the second circuit board when the wearable device is secured to the user's skin; said first temperature sensor is mounted to the first circuit board and the second temperature sensor is mounted to the second circuit board; said third temperature sensor is mounted to the first circuit board and spaced from the first temperature sensor; said fourth temperature sensor is mounted to the second circuit board and spaced from the second temperature sensor; a distance between the first temperature sensor and the second circuit board at least partially defines said air gap; and said thermally conductive element is positioned between the third temperature sensor and a portion of the second circuit board that is adjacent to the fourth temperature sensor. In some implementations, said first circuit board comprises a first surface and a second surface; said second circuit board comprises a first surface and a second surface; said first surface of the first circuit board faces toward the second surface of the second circuit board; said first and third temperature sensors are mounted on the first surface of the first circuit board; said second and fourth temperature sensors are mounted on the first surface of the second circuit board; and said thermally conductive element is positioned between the third temperature sensor and a portion of the second surface of the second circuit board that is adjacent to the fourth temperature sensor. In some implementations, said second circuit board comprises at least one opening positioned between the fourth temperature sensor and a portion of said thermally conductive element, said at least one opening configured to allow thermal energy to pass from the thermally conductive element through the second circuit board and to the fourth temperature sensor.

In some implementations, said first and second temperature sensors are substantially aligned with one another and said third and fourth temperature sensors are substantially aligned with one another. In some implementations, said thermally conductive element comprises a metal strip. In some implementations, said metal strip comprises copper. In some implementations, the first and second circuit boards are arranged to be substantially parallel to one another. In some implementations, said one or more hardware processors are further configured to determine said one or more body temperature values based on: a third comparison between said one or more signals generated by at least one of the first pair of temperature sensors and said one or more signals generated by at least one of the second pair of temperature sensors. In some implementations, said one or more hardware processors are further configured to determine said one or more body temperature values based on: a third comparison between said one or more signals generated by the first temperature sensor and said one or more signals generated by the fourth temperature sensor.

In some implementations, the wearable device comprises a first portion configured to be secured to the user's skin and a second portion configured to removably secure to the first portion, and the one or more hardware processors, the first pair of temperature sensors, and the second pair of temperature sensors are positioned within the second portion of the wearable device. In some implementations, the first portion comprises a frame and a substrate coupled to the frame, the substrate configured to secure to the user's skin, and wherein the second portion comprises a housing. In some implementations, the housing comprises a first shell and a second shell, and wherein the first and second shells are permanently secured together. In some implementations, none of the first temperature sensor, second temperature sensor, third temperature sensor, and fourth temperature sensor contact the user's skin when the wearable device is secured to the user.

For purposes of summarizing the disclosure, certain aspects, advantages, and novel features are discussed herein. It is to be understood that not necessarily all such aspects, advantages, or features will be embodied in any particular implementation of the disclosure, and an artisan would recognize from the disclosure herein a myriad of combinations of such aspects, advantages, or features.

### BRIEF DESCRIPTION OF THE DRAWINGS

Certain features of this disclosure are described below with reference to the drawings. The illustrated implementations are intended to illustrate, but not to limit, the implementations. Various features of the different disclosed implementations can be combined to form further implementations, which are part of this disclosure.

FIGS. 1A-1B illustrate top perspective views of a wearable device in accordance with aspects of this disclosure.

FIG. 1C illustrates a top view of the wearable device of FIGS. 1A-1B in accordance with aspects of this disclosure.

FIG. 1D illustrates a bottom view of the wearable device of FIGS. 1A-1B in accordance with aspects of this disclosure.

FIG. 1E illustrates a side view of the wearable device of FIGS. 1A-1B in accordance with aspects of this disclosure.

FIG. 1F illustrates another side view of the wearable device of FIGS. 1A-1B in accordance with aspects of this disclosure.

FIG. 1G illustrates a front view of the wearable device of FIGS. 1A-1B in accordance with aspects of this disclosure.

FIG. 1H illustrates a back view of the wearable device of FIGS. 1A-1B in accordance with aspects of this disclosure.

FIG. 2 illustrates a schematic block diagram of the wearable device of FIGS. 1A-1B in accordance with aspects of this disclosure.

FIGS. 3A-3B illustrate top perspective views of a hub and a dock of the wearable device of FIGS. 1A-1B separated from one another in accordance with aspects of this disclosure.

FIGS. 4A-4B illustrate top perspective views of the dock of FIGS. 3A-3B in accordance with aspects of this disclosure.

FIG. 4C illustrates a side view of the dock of FIGS. 4A-4B in accordance with aspects of this disclosure.

FIG. 4D illustrates a bottom perspective view of the dock of FIGS. 4A-4B in accordance with aspects of this disclosure.

FIG. 4E illustrates an exploded perspective view of the dock of FIGS. 4A-4B in accordance with aspects of this disclosure.

FIGS. 5A-5B illustrate top perspective views of the hub of FIGS. 3A-3B in accordance with aspects of this disclosure.

FIG. 5C illustrates a bottom perspective view of the hub of FIGS. 5A-5B in accordance with aspects of this disclosure.

FIG. 5D illustrates a side view of the hub of FIGS. 5A-5B in accordance with aspects of this disclosure.

FIGS. 5E-5F illustrate top perspective partially exploded views of the hub of FIGS. 5A-5B in accordance with aspects of this disclosure.

FIGS. 5G-5H illustrate bottom perspective views of a first portion of a housing of the hub of FIGS. 5A-5B in accordance with aspects of this disclosure.

FIGS. 5I-5J illustrate top perspective views of a second portion of the housing of the hub of FIGS. 5A-5B in accordance with aspects of this disclosure.

FIGS. 5K-5L illustrate top perspective views of a portion of the hub of FIGS. 5A-5B in accordance with aspects of this disclosure.

FIGS. 5M-5N illustrate bottom perspective views of the portion of the hub of FIGS. 5K-5L in accordance with aspects of this disclosure.

FIG. 5O illustrates a bottom perspective partially exploded view of the portion of the hub of FIGS. 5K-5L in accordance with aspects of this disclosure.

FIG. 5P illustrates a top perspective view of the portion of the hub of FIGS. 5K-5L and the second portion of the housing of FIGS. 5I-5J in accordance with aspects of this disclosure.

FIG. 5Q illustrates a top view of a circuit board of the hub of FIGS. 5A-5B in accordance with aspects of this disclosure.

FIG. 5R illustrates a top view of another circuit board of the hub of FIGS. 5A-5B in accordance with aspects of this disclosure.

FIG. 5S illustrates a cross-sectional view through the portion of the hub as shown in FIGS. 5E-5F in accordance with aspects of this disclosure.

FIG. 5T illustrates a cross-sectional view through the wearable device as shown in FIG. 1C in accordance with aspects of this disclosure.

FIGS. 6A-6C illustrate alternative implementations for a hub of the wearable device in accordance with aspects of this disclosure.

FIGS. 7A-7F illustrate alternative implementations of portions of a hub of the wearable device in accordance with aspects of this disclosure.

FIG. 8 illustrates a schematic diagram of another implementation of a wearable device in accordance with aspects of this disclosure.

### DETAILED DESCRIPTION

Various features and advantages of this disclosure will now be described with reference to the accompanying figures. The following description is merely illustrative in nature and is in no way intended to limit the disclosure, its application, or uses. This disclosure extends beyond the specifically disclosed implementations and/or uses and obvious modifications and equivalents thereof. Thus, it is intended that the scope of this disclosure should not be limited by any particular implementation described herein. The features of the illustrated implementations can be modified, combined, removed, and/or substituted as will be apparent to those of ordinary skill in the art upon consideration of the principles disclosed herein.

Various wearable devices are disclosed herein which can be utilized to provide improved estimations of internal body temperature (which also may be referred to herein as “core body temperature” or “body temperature”). FIG. 8 shows a schematic diagram of an illustrative wearable device 800. Wearable device 800 can be configured to be secured to a user's skin, for example, via an adhesive material (user can also be referred to herein as “subject” or “wearer”). For example, a portion of wearable device 800 can include an adhesive material (for example, a medical grade adhesive) that can allow wearable device 800 (or a portion thereof) to secure (for example, removably secure) to the user's skin. As another example, in some implementations, wearable device 800 includes one or more substrates coupled to a housing 802 of wearable device 800 and configured to secure wearable device 800 to the user's skin. For example, in some implementations, wearable device 800 can include a substrate that is similar or identical to substrate 250 described elsewhere herein. In some implementations, wearable device 800 includes two separable components (which may also be referred to as “first” and “second” portions of wearable device 800). In some of such implementations, wearable device 800 includes a first component (which may be referred to as a “first portion”) that can secure to skin of a user. Additionally, in some of such implementations, wearable device 800 includes a second component (which may be referred to as a “second portion”) that may include housing 802 and/or any of the features discussed and/or illustrated as being inside and/or connected to housing 802, such as probe 810a and/or probe 810b, circuit boards 804, 806, thermally conductive element 808b, and/or any of temperature sensors T1-T4, each of which are discussed in more detail below. In some of such implementations, such first and second components of wearable device 800 are removably connectable to one another. In some of such implementations, when the first and second components are connected to one another and when the wearable device 800 is in use, at least a portion of the first component is positioned between: housing 802 (or a portion thereof, such as a bottom portion thereof) and the user's skin; probes 810a, 810b and the user's skin; and/or one or more of temperature sensors T1-T4 and the user's skin. In some implementations, when wearable device 800 is in use (for example, secured to the user's skin), no temperature sensors of the wearable device 800 contact skin of the user. In some implementations, when wearable device 800 is in use (for example, placed adjacent the user's skin) and wearable device 800 includes thermally conductive probe 810a and/or 810b, neither of thermally conductive probes 810a, 810b contact skin of the user.

With continued reference to FIG. 8, wearable device 800 can include a housing 802 that can enclose various electronic components thereof. Such housing 802 can be rigid, for example. Wearable device 800 can include one or more temperature sensors, such as one, two, three, four, five, six, seven, eight, nine or ten or more temperature sensors. Wearable device 800 can include one or more pairs of temperature sensors, such as two pairs of temperature sensors (for example, T1 and T2 being a first pair and T3 and T4 being a second pair as shown in FIG. 8). Although FIG. 8 illustrates wearable device 800 having four temperature sensors—represented by T1, T2, T3, and T4—an alternative number and/or arrangement of temperature sensors is possible. Wearable device 800 (or portions thereof) can be configured to operably position temperature sensors T1, T2, T3, T4 in various locations within housing 802 with respect to each other and/or with respect to the user's skin (when wearable device 800 is positioned adjacent and/or secured to the user's skin). For example, wearable device 800 can include structure(s) that operably position temperature sensors T1, T2, T3, T4. In some implementations, wearable device 800 includes one or more circuit boards within housing 802 that can mount temperature sensors T1, T2, T3, T4. For example, as shown in FIG. 8, wearable device 800 can include circuit boards 804, 806. Circuit boards 804, 806 can be spaced apart from one another within housing 802, for example, by a gap. In some implementations, wearable device 800 includes a battery which is positioned between the first and second circuit boards 804, 806 (for example, that can be similar or identical to battery 332 discussed elsewhere herein). In some implementations, circuit boards 804, 806 are oriented substantially parallel to one another within housing 802, as shown. In some implementations, T1 and T2 are substantially aligned with one another, for example, along an axis 805 that can be substantially parallel to axis 1. Additionally or alternatively, in some implementations, T3 and T4 are substantially aligned with one another, for example, along an axis 807 that can be substantially parallel to axis 1. In some implementations, such axes 805, 807 are substantially parallel to one another. In some implementations, axes 805, 807 are spaced from one another by a distance 809. In some implementations, T1 and T2 are substantially aligned with one another (along axis 805) and are both spaced away from T3 and T4, which are substantially aligned with one another (along axis 807). In some implementations, T1 and T2 are spaced from one another along axis 805 by a distance 811, which can include a thickness 815 of circuit board 804. In some implementations, T3 and T4 are spaced from one another along axis 807 by a distance 813, which can include thickness 815 of circuit board 804. In some implementations, distance 809 is greater than distance 811 and/or distance 813. As shown in FIG. 8, T2 and T4 can be spaced from an interior surface of the housing 802 (for example, a top interior surface of housing 802) by gaps 807a, 807b. In some implementations, a thermally conductive material is positioned between T2 and such interior surface of housing 802 and/or between T4 and such interior surface of housing 802. Such thermally conductive material can be, for example, thermal putty (for example, a ceramic filled silicone sheet).

In some implementations, circuit boards 804, 806 can include one or more openings adjacent locations wherein temperature sensors T1, T2, T3, T4 are mounted to circuit boards 804, 806. For example, as shown in FIG. 8, circuit board 804 can include an opening 804a adjacent T2 and an opening 804b adjacent T4, and/or circuit board 806 can include an opening 806a adjacent T1 and an opening 806b adjacent T3. In some implementations in which T1 and T2 are substantially aligned and T3 and T4 are substantially aligned, openings 804a, 806a can be substantially aligned with T1 and T2, and openings 804b, 806b can be substantially aligned with T3 and T4. Such openings 804a, 806a, 804b, 806b can help at least partially define a thermal flow path from and/or between T1 and T2 and a thermal flow path from and/or between T3 and T4. While openings 804a, 806a, 804b, 806b are each illustrated as a single opening, each of said openings 804a, 806a, 804b, 806b can be formed as a plurality of openings positioned in circuit boards 804, 806 at least partially adjacent T1, T2, T3, T4. Additionally, although openings 804a, 806a, 804b, 806b are shown as having widths substantially equal to widths of T1-T4 , this is not intended to be limiting. The illustrated openings 804a, 806a, 804b, 806b (each of which may comprise a plurality of openings) can have smaller widths than that shown in FIG. 8. Any of openings 804a, 806a, 804b, 806b (each of which may comprise a plurality of openings) can be positioned in portions of circuit boards 804, 806 adjacent to T1-T4. Openings 804a, 806a, 804b, 806b can allow thermal energy to flow through circuit boards 804, 806 to T1-T4 , for example. In some implementations, one or more of such openings 804a, 806a, 804b, 806b are filled with a thermally conductive material. Alternatively, in some implementations, one or more of such openings 804a, 806a, 804b, 806b are not filled with a thermally conductive material (for example, are left “void”). In some implementations, opening 804a (which may comprise a plurality of openings) is not filled with a thermally conductive material but one or more of openings 804b, 806a, and/or 806b (each of which may comprise a plurality of openings) are filled with a thermally conductive material. Such thermally conductive material can include a metallic material, such as copper.

In some implementations, wearable device 800 includes thermally conductive probes 810a, 810b. Thermally conductive probes 810a, 810b can be made of a thermally conductive material (for example, a metallic material). In some implementations, probes 810a, 810b are rigid. Thermally conductive probes 810a, 810b (for example, ends thereof) can be positioned and/or secured to a surface of circuit board 806 adjacent T1 and T3 (for example, a “bottom” surface given the view shown in FIG. 8). In some variants, thermally conductive probes 810a, 810b extend through openings 806a, 806b, where such openings 806a, 806b are sized and/or shaped to receive thermally conductive probes 810a, 810b. Advantageously, thermally conductive probes 810a, 810b can transmit thermal energy from the user (for example, thermal energy radiating from the user's skin) to and/or toward T1 and T3, and such thermal energy can flow through openings 806a, 806b to T1 and T3. Although ends of thermally conductive probes 810a, 810b are illustrated as contacting the user's skin in FIG. 8, in some implementations, thermally conductive probes 810a, 810b do not contact the user's skin when the wearable device 800 is secured to the user. For example, as discussed previously, in some implementations, wearable device 800 includes one or more substrates that are configured to be positioned between the user's skin and the thermally conductive probes 810a, 810b when the wearable device 800 is secured to the user. In some implementations, housing 802 includes openings that allow the thermally conductive probes 810a, 810b to extend through the housing 802. Such openings in housing 802 can be positioned along an exterior portion of housing 802 near (for example, underneath) openings 806a, 806b in circuit board 806.

FIG. 8 schematically illustrates flow of thermal energy (represented by Qb) from a user's interior to and/or between the user's skin. As mentioned previously, the user's internal body temperature (represented by Tb in FIG. 8) is an important physiological parameter indicative of the user's condition. However, as also discussed above, it is often difficult to noninvasively approximate Tb based on measuring skin temperature. Various aspects of wearable device 800 can allow for improved noninvasive approximations of Tb.

As shown in FIG. 8, thermal energy flowing from the user's body flows through the user's skin. When wearable device 800 is placed and/or secured to the user's skin, such thermal energy can be transmitted by the thermally conductive probes 810a, 810b to and/or toward T1 and T3, for example, via openings 806a, 806b in circuit board 806. A thermally insulative material can be positioned at least partially between T1 and T2, for example, between T1 and a portion of circuit board 804 that is adjacent to T2. For example, in some implementations, an air gap 808a is present between T1 and circuit board 804, opening 804a, and/or T2, as shown. Additionally, a thermally conductive element 808b can be positioned at least partially between T3 and T4, for example, between T3 and circuit board 804, opening 804b, and/or T4, as shown. T2 and T4, due in part to their positioning in housing 802 and relative to the user's skin when wearable device 800 is secured to the skin, can be more responsive to ambient temperatures (for example, temperature outside housing 802 in the surrounding environment which is represented by Ta in FIG. 8). Additionally, T1 and T3 can be more responsive to the user's temperature since they are positioned closer to the user's skin when wearable device 800 is secured to the user. Although T1 and T3 may, in some implementations, be positioned substantially the same distance from the user's skin when wearable device 800 is secured to the user, temperature values determined from T1 and T3 or determined from signal(s) generated from T1 and T3 can be different at least because of the presence of air gap 808a and thermally conductive element 808b.

Such configurations can facilitate a variety of different temperature measurement determinations using T1, T2, T3, and T4. Further, such configurations can be utilized to compare differences between one or more of temperature sensors T1, T2, T3, T4 and/or compare temperature gradients between T1 and T2, T3 and T4, and/or other gradients (for example, T1 and T3, T2 and T4, among others). Because thermal properties of air gap 808a and thermally conductive element 808b may be known, relationships between temperature values determined using T1, T2, T3, T4 can be utilized to approximate internal body temperature Tb, despite the existence of thermal properties of the user's skin (which typically decrease the accuracy of body temperature estimations in conventional temperature measurement devices). For example, in some implementations, wearable device 800 (for example, one or more processors of wearable device 800) can receive one or more signals from each of T1, T2, T3, T4 and determine one or more body temperature values based on comparisons of such signals. For example, wearable device 800 (for example, one or more processors of wearable device 800) can: receive one or more signals generated by T1 and T2 responsive to detected thermal energy; compare the one or more signals generated by T1 and T2; receive one or more signals generated by T3 and T4 responsive to detected thermal energy; compare the one or more signals generated by T3 and T4; and determine one or more body temperature values of the user based on at least such comparisons. In some implementations, wearable device 800 (for example, one or more processors of wearable device 800) determines the one or more body temperature values of the user based additionally on: a comparison between one or more signals generated by T1 and one or more signals generated by T4; a comparison between one or more signals generated by T2 and one or more signals generated by T3; a comparison between one or more signals generated by T1 and one or more signals generated by T3; and/or a comparison between one or more signals generated by T2 and one or more signals generated by T4.

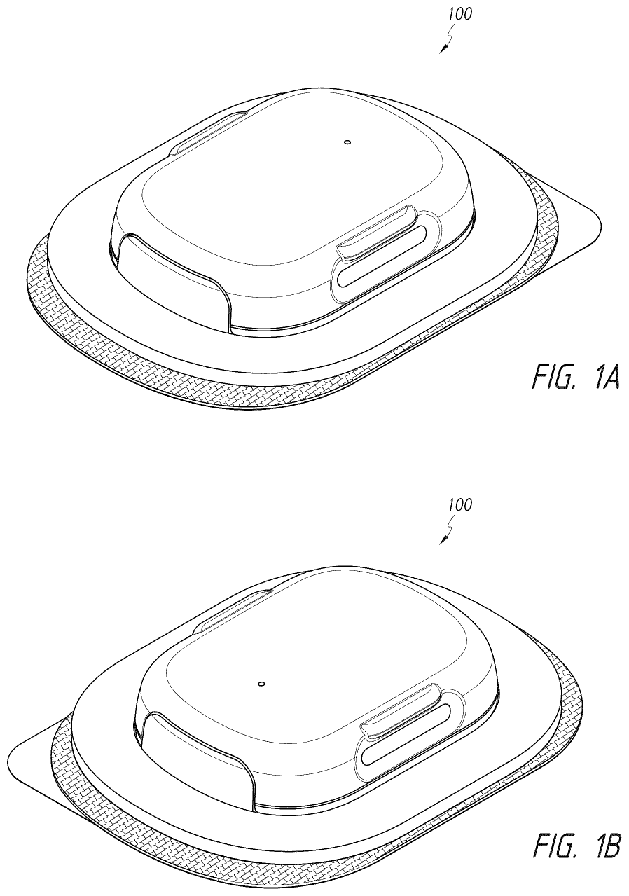

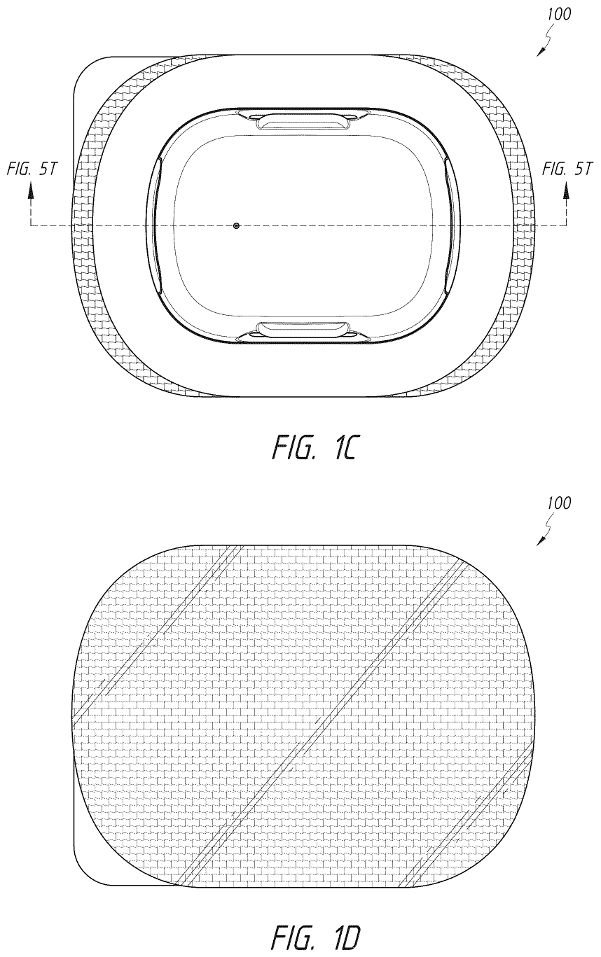

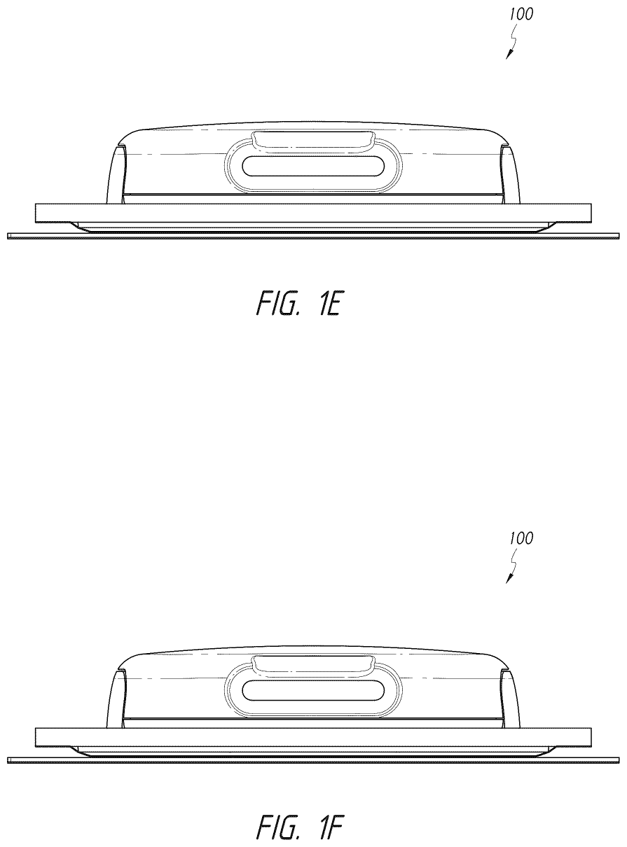

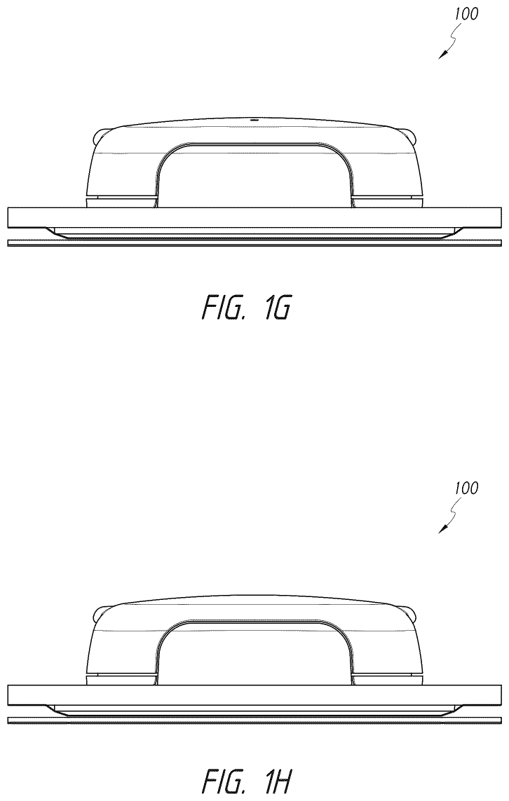

FIGS. 1A-1H illustrate various views of a wearable device 100 that can measure and/or monitor one or more physiological parameters of a subject, as discussed further below. The wearable device 100 can secure to a portion of a subject's body, such as a torso, chest, back, arm, neck, leg, under the arm (e.g., armpit), among other portions of the subject's body. The wearable device 100 can secure (for example, removably secure) to skin of a subject and continuously and/or noninvasively measure the subject's temperature using one or more temperature sensors. Additionally, as discussed below, the wearable device 100 can continuously or periodically wirelessly transmit temperature data of the subject to a separate device.

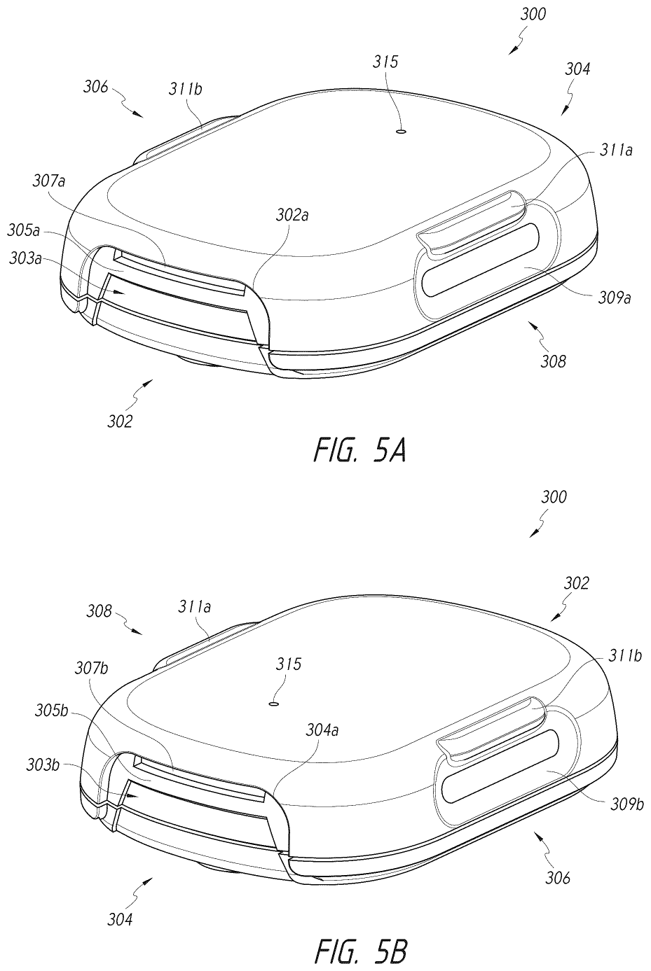

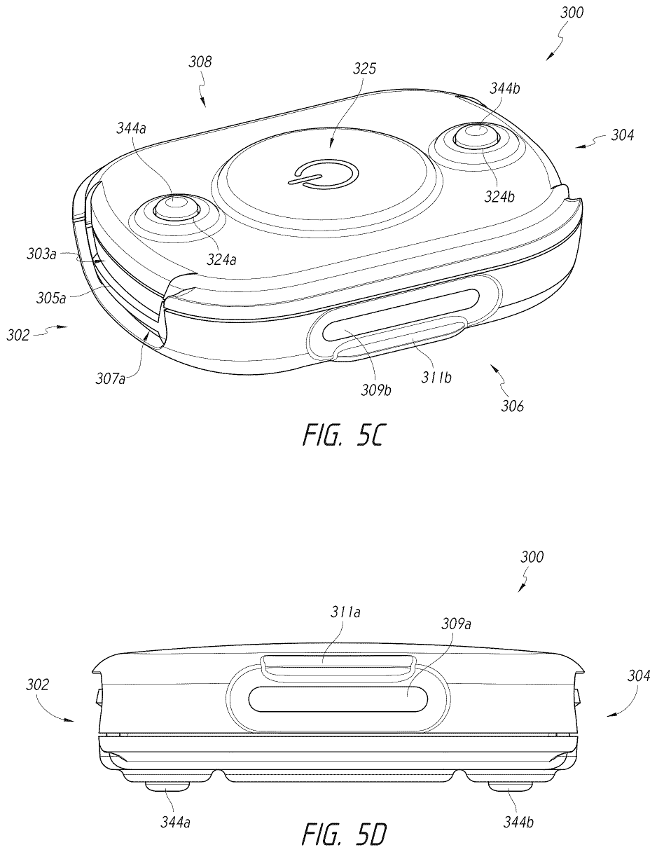

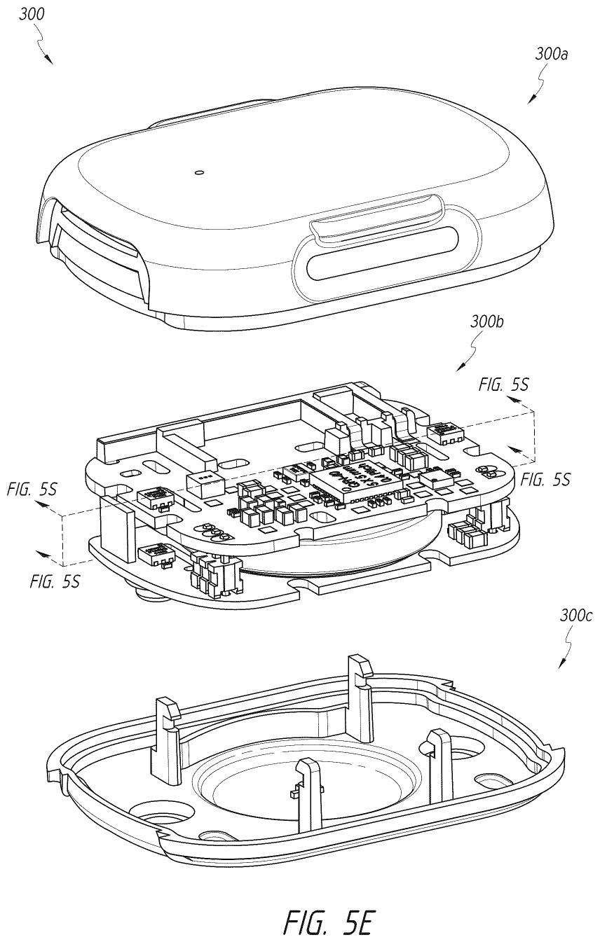

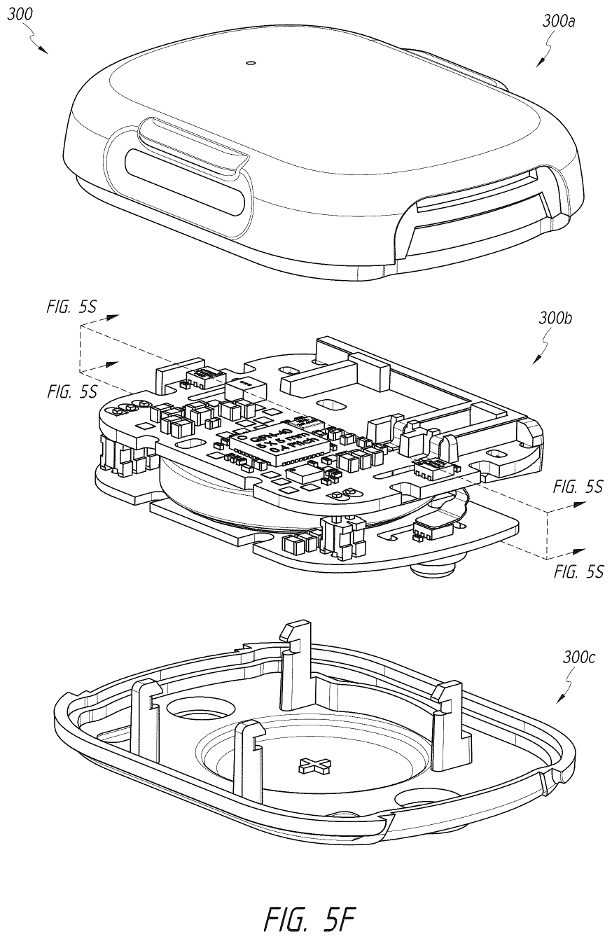

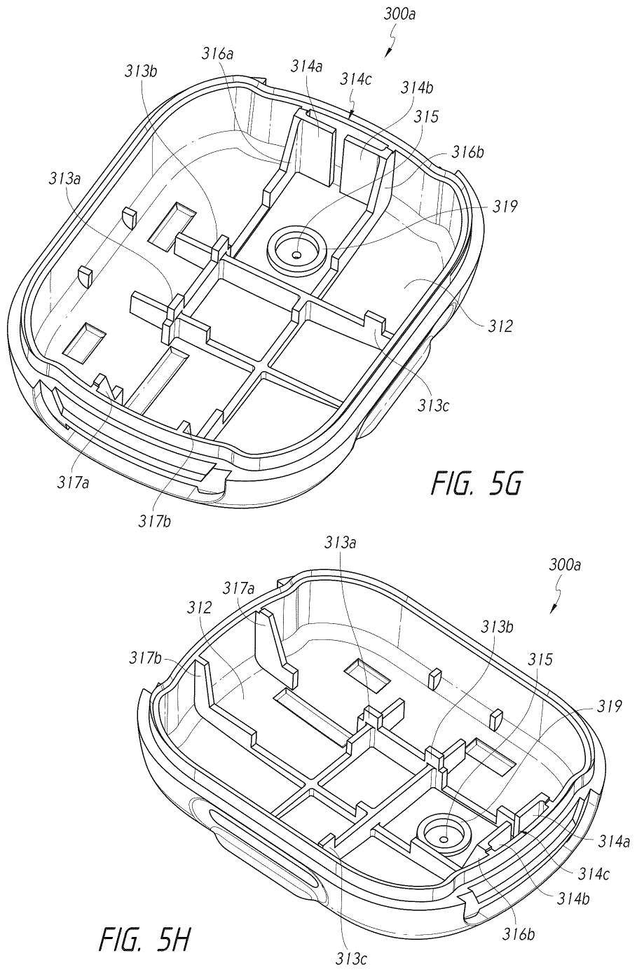

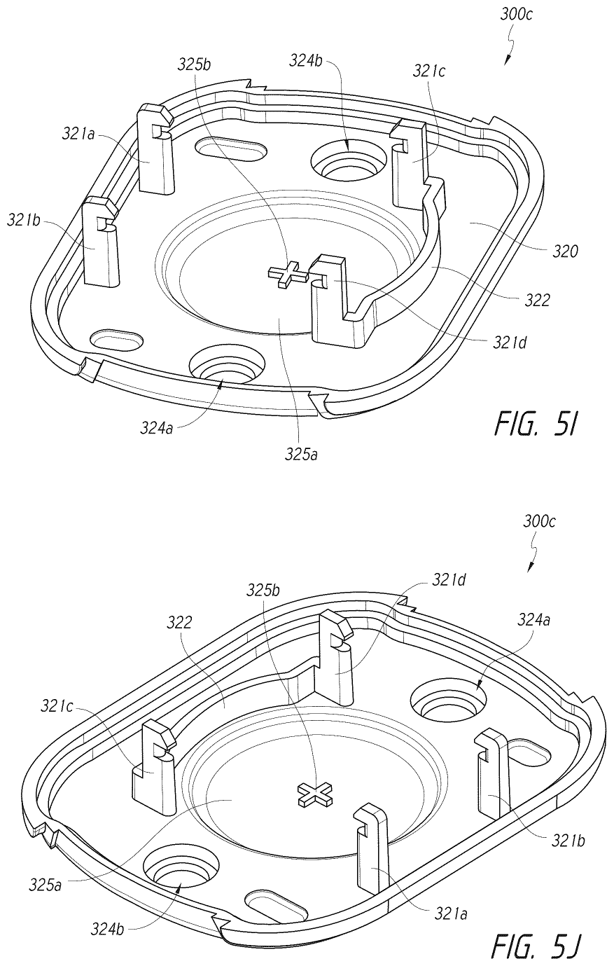

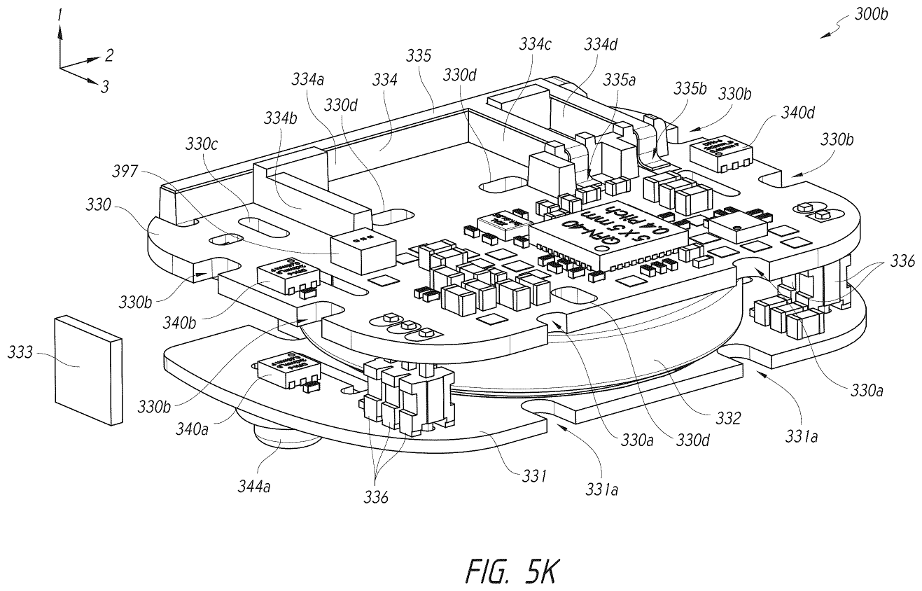

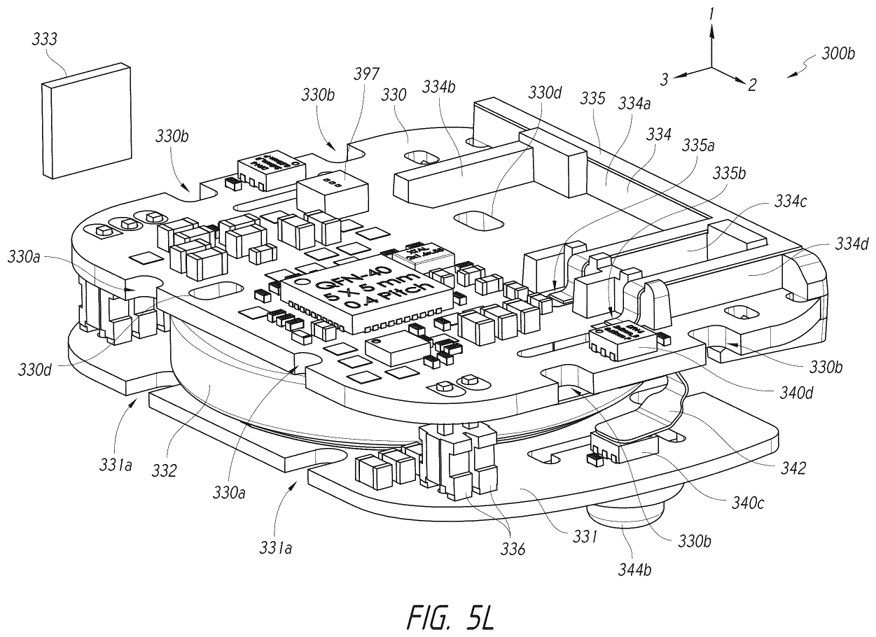

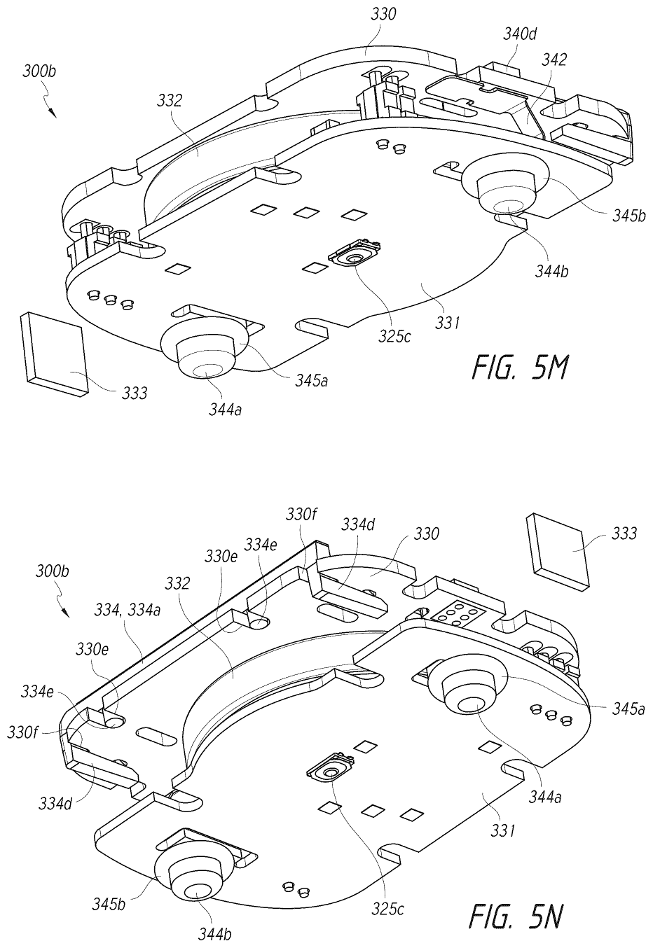

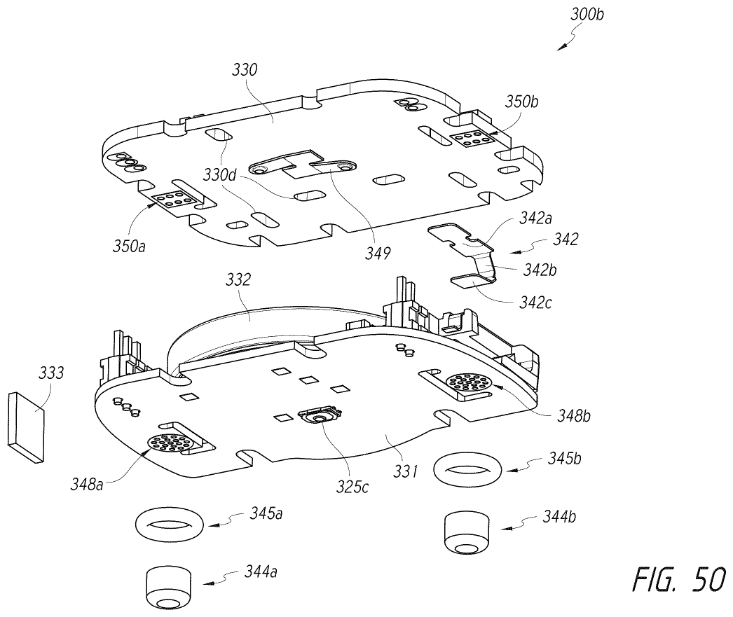

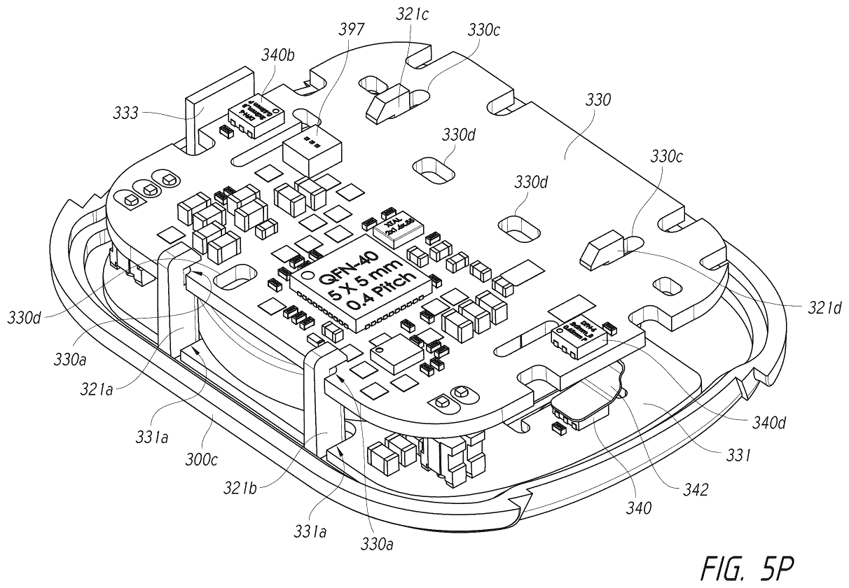

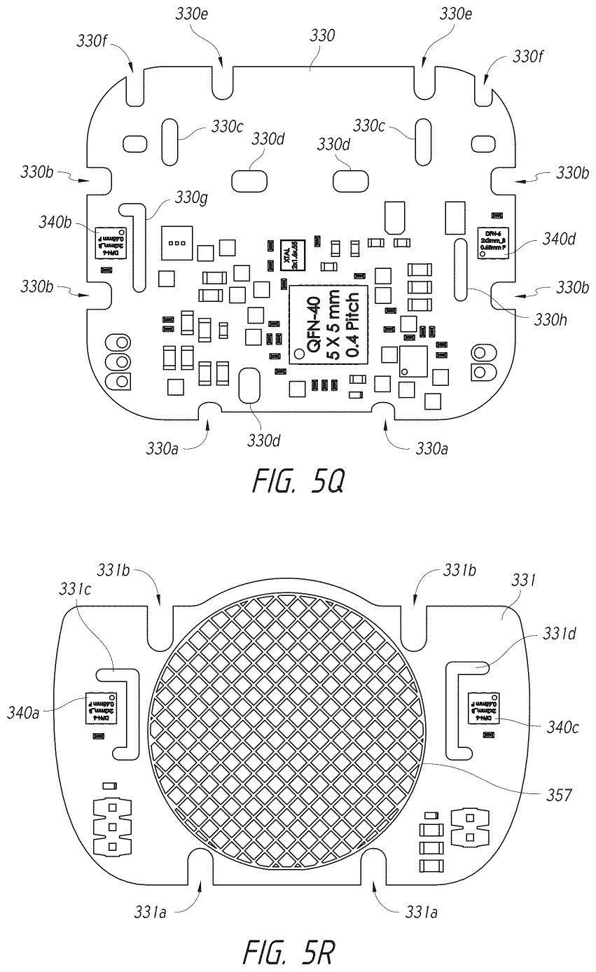

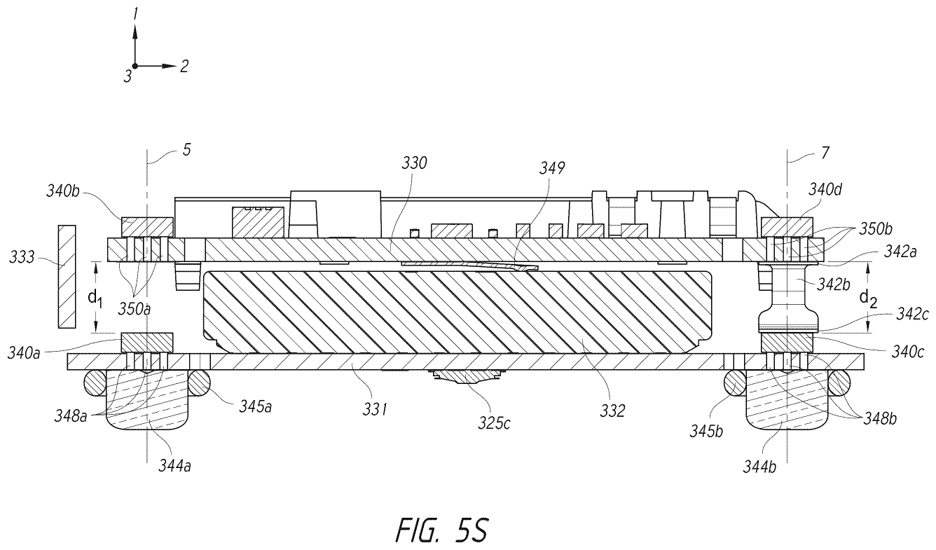

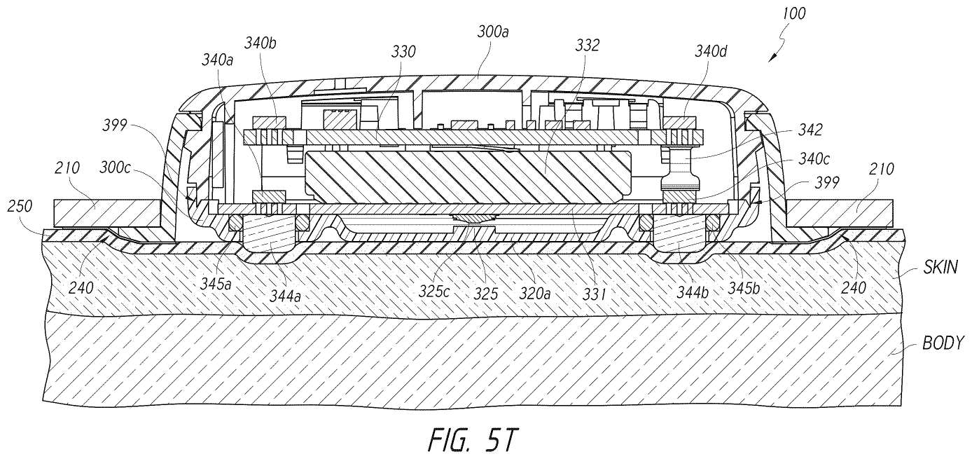

FIG. 5T, discussed in more detail below, illustrates an example cross-section taken through wearable device 100 when wearable device 100 is secured to skin of a subject. As illustrated in FIG. 5T and as discussed further below, wearable device 100 can include thermally conductive probes 344a, 344b that extend toward the subject's skin and transmit thermal energy from the skin in a direction towards temperature sensors of wearable device 100 (such as temperature sensors 340a, 340c discussed further below). As also discussed below, the thermally conductive probes 344a, 344b can indirectly contact (for example, via substrate 250) and/or apply pressure to the subject's skin, which can facilitate thermal transmissivity. In some implementations, the thermally conductive probes 344a, 344b do not contact the subject's skin when wearable device 100 is secured to the subject. For example, the substrate 250 can be positioned between the thermally conductive probes 344a, 344b and the subject's skin when wearable device 100 is secured to the subject as shown.

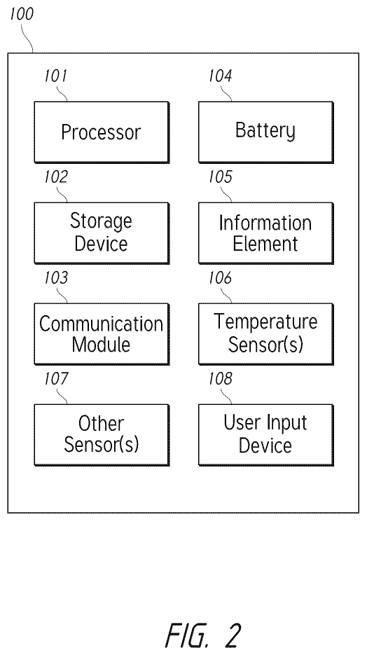

FIG. 2 illustrates an example schematic block diagram of wearable device 100. As shown, wearable device 100 can include a processor 101, a storage device 102, a communication module 103, a battery 104, an information element 105, and/or one or more temperature sensors 106. The processor 101 can be configured, among other things, to process data, execute instructions to perform one or more functions, and/or control the operation of wearable device 100. For example, the processor 101 can process physiological data obtained from wearable device 100 and can execute instructions to perform functions related to storing and/or transmitting such physiological data. For example, the processor 101 can process data received from one or more temperature sensors 106 and/or one or more other sensors 107 and can execute instructions to perform functions related to storing and/or transmitting such received data. Additionally or alternatively, processor 101 can process raw data (for example, signal(s)) generated by the one or more temperature sensors 106 and determine body temperature value(s). In some implementations, wearable device 100 includes a plurality of processors (such as two, three, four, or five or more) that can carry out any of the functions described herein with respect to processor 101 (or any of the other processors described herein).

The storage device 102 can include one or more memory devices that store data, including without limitation, dynamic and/or static random access memory (RAM), programmable read-only memory (PROM), erasable programmable read-only memory (EPROM), electrically erasable programmable read-only memory (EEPROM), and the like. Such stored data can be processed and/or unprocessed physiological data obtained from wearable device 100, for example.

The communication module 103 can facilitate communication (via wired and/or wireless connection) between wearable device 100 (and/or components thereof) and separate devices, such as separate monitoring and/or mobile devices. For example, the communication module 103 can be configured to allow wearable device 100 to wirelessly communicate with other devices, systems, and/or networks over any of a variety of communication protocols. The communication module 103 can be configured to use any of a variety of wireless communication protocols, such as Wi-Fi (802.11x), Bluetooth®, ZigBee®, Z-wave®, cellular telephony, infrared, near-field communications (NFC), RFID, satellite transmission, proprietary protocols, combinations of the same, and the like. The communication module 103 can allow data and/or instructions to be transmitted and/or received to and/or from wearable device 100 and separate computing devices. The communication module 103 can be configured to transmit (for example, wirelessly) processed and/or unprocessed physiological or other information to separate computing devices, which can include, among others, a mobile device (for example, an iOS or Android enabled smartphone, tablet, laptop), a desktop computer, a server or other computing or processing device for display and/or further processing, among other things. Such separate computing devices can be configured to store and/or further process the received physiological and/or other information, to display information indicative of or derived from the received information, and/or to transmit information—including displays, alarms, alerts, and notifications—to various other types of computing devices and/or systems that may be associated with a hospital, a caregiver (for example, a primary care provider), and/or a user (for example, an employer, a school, friends, family) that have permission to access the subject's data. As another example, the communication module 103 of wearable device 100 can be configured to wirelessly transmit processed and/or unprocessed obtained physiological information and/or other information (for example, motion and/or location data) to a mobile phone which can include one or more hardware processors configured to execute an application that generates a graphical user interface displaying information representative of the processed or unprocessed physiological and/or other information obtained from wearable device 100. The communication module 103 can be embodied in one or more components that are in communication with each other. The communication module 103 can comprise a wireless transceiver and/or a near field communication (NFC) component. In some implementations, communication module 103 is embodied in an antenna (such as antenna 335) and/or an NFC chip (such as NFC chip 333).

Wearable device 100 can include a battery 104. The battery 104 can provide power for hardware components of wearable device 100 described herein. The battery 104 can be, for example, battery 332, described in more detail below. The battery 104 can be, for example, a lithium battery. Additionally or alternatively, wearable device 100 can be configured to obtain power from a power source that is external to wearable device 100. For example, wearable device 100 can include or can be configured to connect to a cable which can itself connect to an external power source to provide power to wearable device 100.

Wearable device 100 can include an information element 105. The information element 105 can be a memory storage element that stores, in non-volatile memory, information used to help maintain a standard of quality associated with wearable device 100. Illustratively, the information element 105 can store information regarding whether wearable device 100 has been previously activated and whether wearable device 100 has been previously operational for a prolonged period of time, such as, for example, four hours, one day, two days, five days, ten days, twenty days. The information stored in the information element 105 can be used to help detect improper re-use of wearable device 100, for example.

With continued reference to FIG. 2, wearable device 100 can include a user input device 108 that can allow a subject (or a caregiver) to interact with wearable device 100. User input device 108 can be utilized to transition wearable device 100 from a non-operational mode to an operational mode and vice versa, for example, or carry out other actions. Button 325 (see FIG. 5C), discussed further below, can be an implementation of user input device 108. As also discussed further below, button 325 can include an actuator portion (for example, pad 325a and protrusion 325b in FIGS. 5I-5J) and a switch (such as switch 325c in FIGS. 5M-5N).

As shown in FIG. 2, wearable device 100 can include one or more temperature sensors 106 that can be used to continuously or periodically obtain temperature data of a subject. Advantageously, in some implementations, the processor 101 can compare temperature data from more than one temperature sensor 106 to more accurately determine body temperature of the subject. In some variants, wearable device 100 includes one or more temperature sensors 106 and also includes one or more other sensors 107, such as one or more of an accelerometer, a gyroscope, a magnetometer, an oximetry sensor, a moisture sensor, an impedance sensor, an acoustic/respiration sensor, and/or an ECG sensor. In some implementations, wearable device 100 includes one or more temperature sensors 106 and does not include an accelerometer, a gyroscope, a magnetometer, an oximetry sensor, a moisture sensor, an impedance sensor, an acoustic/respiration sensor, and/or an ECG sensor, which can advantageously help conserve battery and processing power and preserve processing capabilities of wearable device 100 where continuous or periodic body temperature values are being determined and/or transmitted. In some implementations, the only type of physiological parameter measured and/or monitored by wearable device 100 is body temperature. Temperature sensors 340a, 340b, 340c, 340d, each of which are discussed in more detail below, can be implementations of the one or more temperature sensors 106. The processor 101 of wearable device 100 can be configured to process obtained physiological information. For example, the processor 101 can be configured to determine body temperature of a user by utilization of one or more temperature sensors 106. Each of the one or more temperature sensors 106 can generate one or more signals responsive to detected thermal energy and such one or more signals can be received by processor 101 for determination of body temperature value(s). Additionally or alternatively, each of the one or more temperature sensors 106 can determine temperature values and transmit such temperature values to processor 101 for determination of body temperature value(s). The one or more temperature sensors 106 can be thermistors or integrated circuit (IC) temperature sensors, for example.

In some implementations, portions of wearable device 100 can be removable from each other. For example, in some implementations, wearable device 100 includes two separable components (which may also be referred to as “first” and “second” portions of wearable device 100). In some of such implementations, wearable device 100 includes a first component (which may be referred to as a “first portion”) that can secure (for example, removably secure) to skin of a subject. Additionally, in some of such implementations, wearable device 100 includes a second component (which may be referred to as a “second portion”). Such second portion can include various electronic components, for example, any of those discussed with respect to FIG. 2 and/or any of those discussed with respect to hub 300 further below. Such first portion can include one or more substrates configured to adhere (for example, removably adhere) to skin. In some implementations, such first portion does not include any electronic components, for example, where any and all electronic components of wearable device 100 are contained in the second portion of wearable device 100. Such first and second portions can be configured to mechanical removably secure to one another. In some implementations, such first and second portions are configured to be difficult to detach from one another if the first portion is secured to the user. In some implementations, the intended service lives of the first and second portions are different. For example, the intended service life of the first portion can be less than the intended service life of the second portion, such as where the first portion includes one or more substrates that secure to the user's skin and where the second portion includes electronic components of wearable device 100. In such implementations, the first portion can be disposed of and replaced and the second portion can be secured with a new first portion. This is advantageous where substrate(s) of such first portion lose integrity and/or become degraded after an amount of time than is less than a battery life of the second portion. Implementations of such first and second portions of wearable device 100 can be dock 200 and hub 300 (respectively) discussed further below.

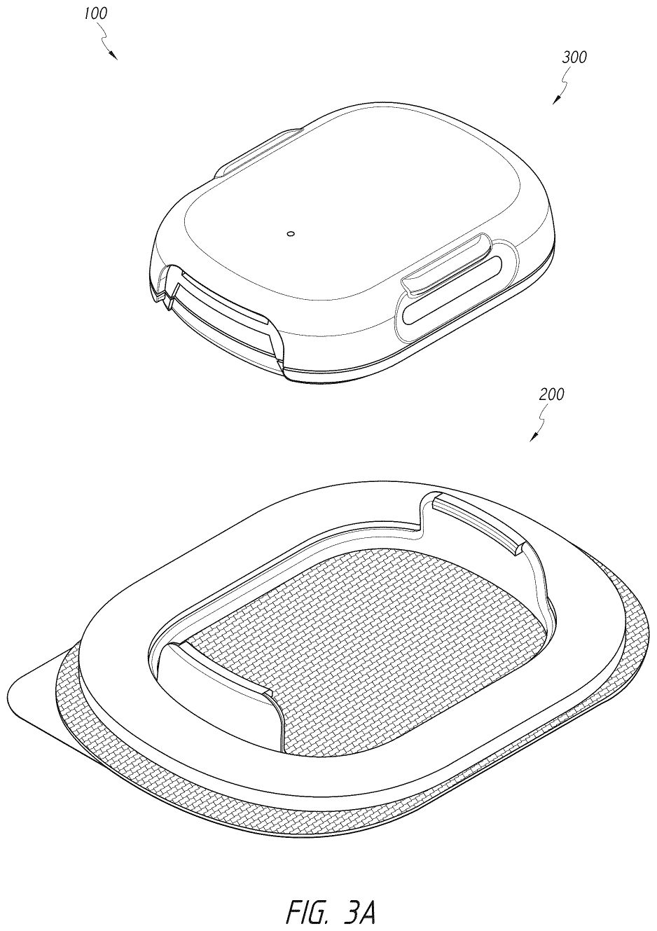

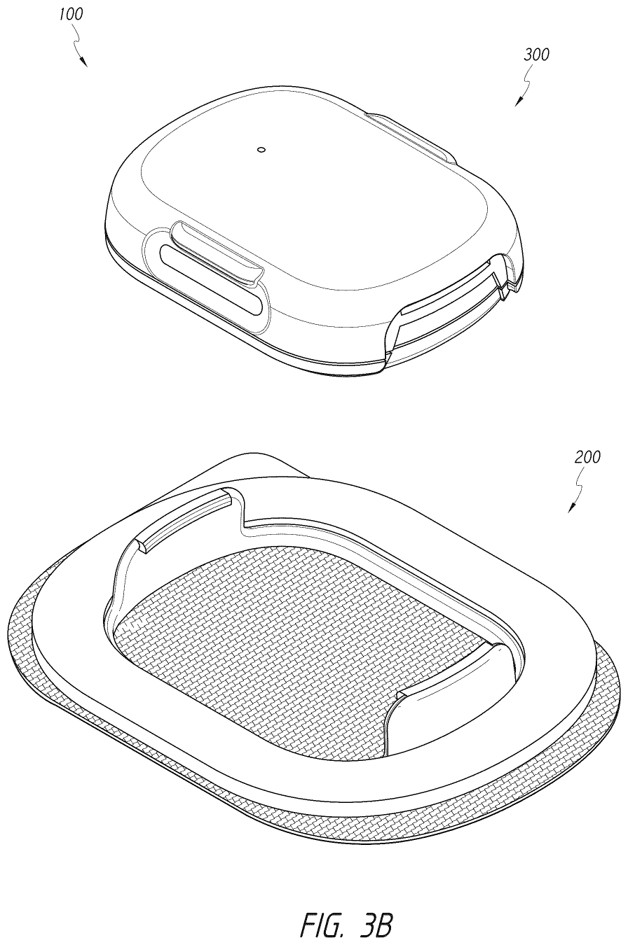

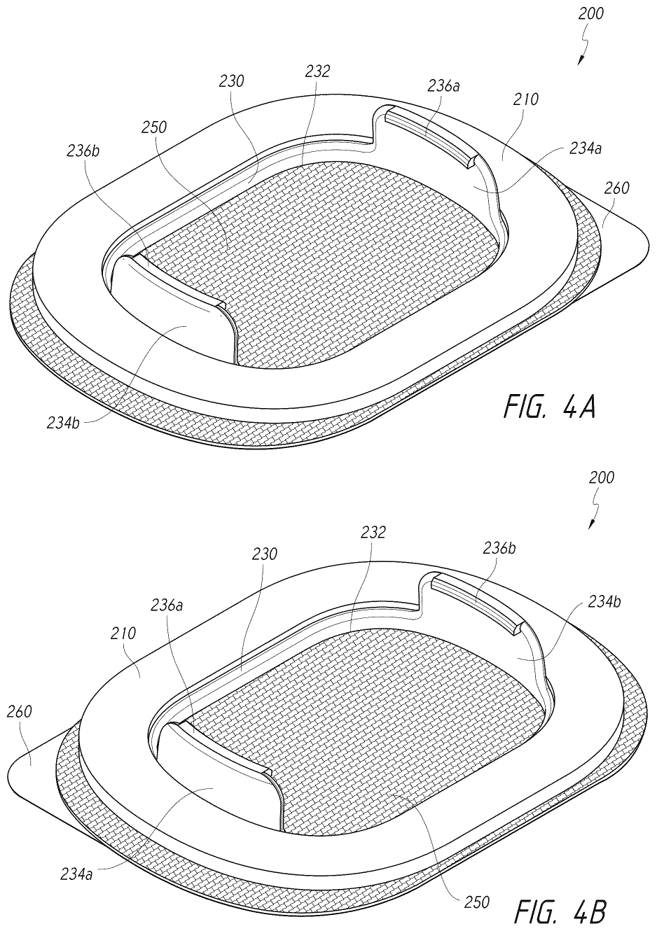

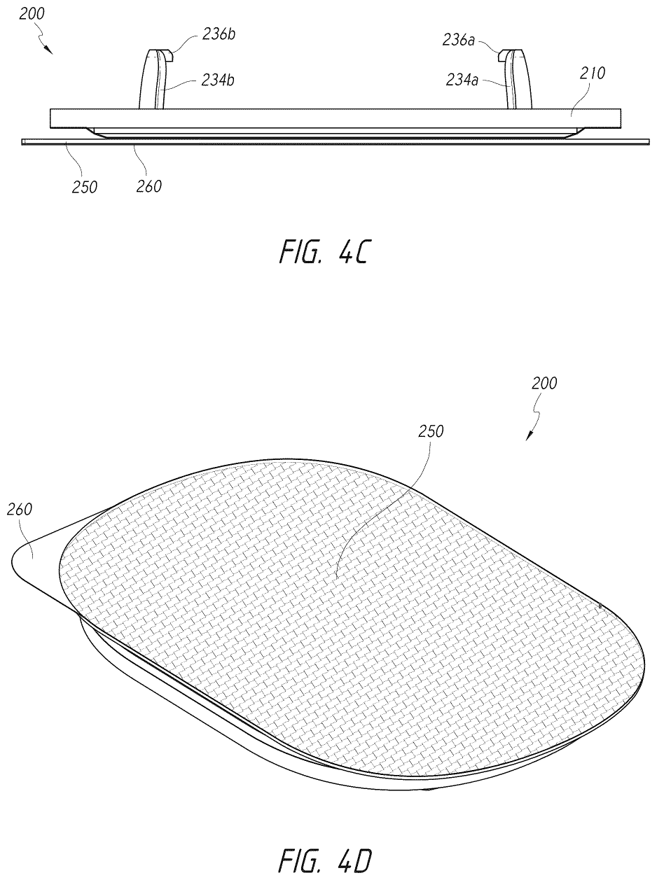

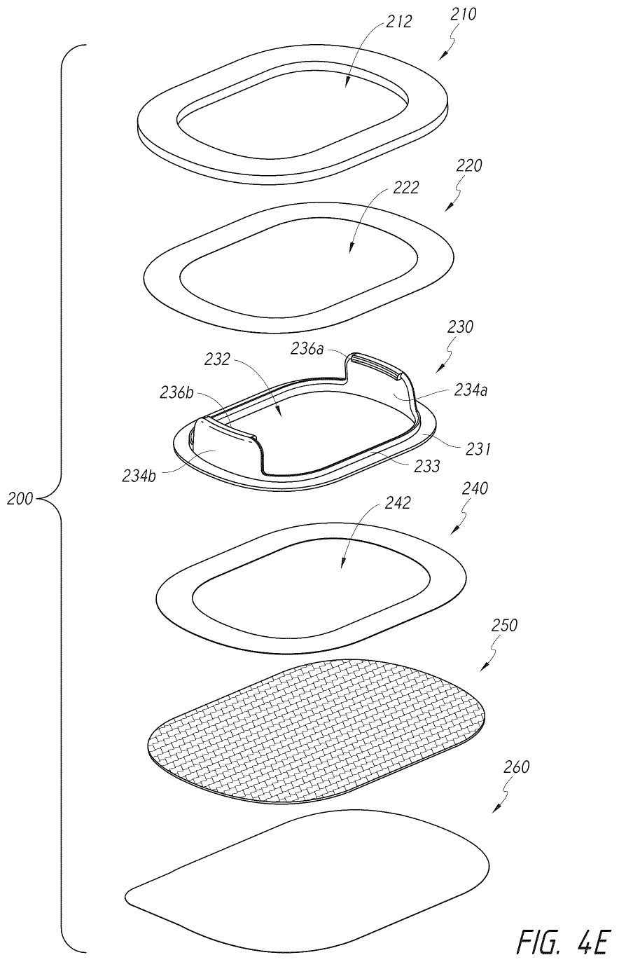

FIGS. 3A-3B illustrate perspective views of dock 200 and hub 300 of wearable device 100 separated from one another, whereas FIGS. 1A-1H illustrates dock 200 and hub 300 attached to one another. FIGS. 4A-4E illustrate various views of dock 200 or portions thereof, while FIGS. 5A-5S illustrate various views of hub 300 or portions thereof.

FIGS. 4A-4B illustrate perspective views of dock 200. FIG. 4C illustrates a first side view of dock 200, which can be a mirror image of an opposite second side view of dock 200. FIG. 4D illustrates a bottom perspective view of dock 200. FIG. 4E illustrates an exploded view of dock 200.

With reference to FIGS. 4A-4E, dock 200 can include a frame 230 and one or more substrates coupled to frame 230, such as any of substrates 210, 220, 240, 250, and/or 260, each of which are discussed in more detail below. With reference to FIG. 4E, frame 230 can include a rim 231 and an opening 232. Rim 231 can define a perimeter of the frame 230. Rim 231 can have a rounded shape. Frame 230 can be configured to removably secure to hub 300, for example, to a housing of hub 300. Frame 230 can include one or more arms which are configured to engage with portions of the housing of the hub 300. For example, frame 230 can include arms 234a, 234b that can extend outward from rim 231. Arm 234a can be positioned at a first end of frame 230 and arm 234b can be positioned at a second end of frame 230 that is opposite of such first end of frame 230. Arms 234a, 234b can extend along a portion of rim 231, for example, less than an entire perimeter of rim 231. Arms 234a, 234b can extend generally perpendicular to rim 231 (for example, a plane defined by rim 231) and/or can extend generally perpendicular to opening 232 (for example, a plane defined by opening 232). In some implementations, frame 230 comprises a wall 233 that extends outward from (for example, generally perpendicular to) rim 231. Wall 233 can have a height that is less than arms 234a, 234b, as shown in FIG. 4E. In some implementations, arms 234a, 234b are curved along lengths (which may also be referred to as widths) thereof. For example, arms 234a, 234b can be curved to correspond with a curved shape of rim 231 at first and second ends of frame 230.

Arms 234a, 234b can be configured to engage with portions of a housing of hub 300 to facilitate securement (for example, removable securement) of the dock 200 and hub 300. As shown, arms 234a, 234b can include protrusions 236a, 236b. Arm 234a can include a first surface (which may be referred to as an “inward surface”) and a second surface (which may be referred to as an “outward surface”) opposite the first surface and arm 234b can include a first surface (which may be referred to as an “inward surface”) and a second surface (which may be referred to as an “outward surface”) opposite the first surface of arm 234b. The first surfaces of arms 234a, 234b can face at least partially toward each other (for example, can face in an inward direction of frame 230) and the second surfaces of the arms 234a, 234b can face away from one other. Protrusions 236a, 236b can extend outward from such respective inward surfaces of arms 234a, 234b and at least partially in a direction toward one another and/or toward an interior of frame 230. Protrusion 236a can extend along a portion of a length of arm 234a and protrusion 236b can extend along a portion of a length of arm 236a. While the figures illustrate protrusions 236a, 236b having a continuous length, in some variants, one or both of arms 234a, 234b include a plurality of protrusions separated from one another, for example, in a location such as that shown with respect to protrusions 236a, 236b. Protrusions 236a, 236b can engage recesses 307a, 307b of hub 300 as discussed in more detail below, which can facilitate securement of the hub 300 and the dock 200. With reference to FIG. 4C, protrusions 236a, 236b can have a beveled or chamfered edge on free ends thereof, which can facilitate movement along a portion of ends 302, 304 of the hub 300 and positioning within recesses 307a, 307b as explained in more detail below. Although arms 234a, 234b are shown as having protrusions 236a, 236b and hub 300 is shown as having recesses 307a, 307b, in some variants, arms 234a, 234b have recesses instead of protrusions 236a, 236b and hub 300 has protrusions instead of recesses 307a, 307b.

As also described further below, in some implementations, arms 234a, 234b can be configured to move when forces are applied to dock 200, which can facilitate removal of protrusions 236a, 236b from recesses 307a, 307b of hub 300 (see FIGS. 5A-5B). For example, in some implementations, application of opposing forces on opposite sides of the dock 200 (which extend between ends of dock 200 where arms 234a, 234b are located) can cause arms 234a, 234b to move from a first position (as shown in FIG. 4C) to a second position where arms 234a, 234b are positioned farther from each other than when in the first position. In some implementations, application of generally opposing forces on opposite ends of the dock 200 (where arms 234a, 234b are located) can cause arms 234a, 234b to move from the first position to the second position. In such second position, arm 234a, 234b can be flexed outward from one another (for example, to the “right” and “left” given the view shown in FIG. 4C). Such configuration can move the protrusions 236a, 236b out of recesses 307a, 307b, thereby allowing hub 300 to be removed from dock 200. In some implementations, dock 200 does not include a clip or other structure(s) that can be actuated by a user to disengage the protrusions 236a, 236b from recesses 307a, 307b. For example, in some implementations, in order for the hub 300 and dock 200 to be removed from each other, a portion or portions of the dock 200 (for example, the frame 200) must be deformed (for example, outward flexing of arms 234a, 234b). In some cases, such configurations make it difficult to remove hub 300 and dock 200 from one another when dock 200 is secured to the user's skin.

Such configurations can be advantageous to prevent users from separating the hub 300 and dock 200 from one another and interfering with operation of wearable device 100. As discussed further below, the hub 300 can include a button that allows the hub 300 to be transitioned from a non-operational mode to an operational mode, and in some implementations, such button is inaccessible when the hub 300 and dock 200 are coupled together. Such configurations can also inhibit users from intentionally or unintentionally interfering with operation of wearable device 100 (for example, shutting it off). For example, in some implementations, in order for wearable device 100 to be turned off, dock 200 and hub 300 must be removed (while coupled together) from the user's skin, arms 234a, 234b must be flexed outward (thereby removing protrusions 236a, 236b from recesses 307a, 307b of hub 300), and hub 300 must be decoupled from dock 200. In such configurations, it may be difficult for a user to carry out such actions when wearable device 100 is secured to the user's own skin, but such actions may be performed by a caregiver, which may be desirable in certain situations.

As mentioned above, FIG. 4E illustrates an exploded view of dock 200. Dock 200 can include one or more substrates that can secure and/or secure to other portions of dock 200 and/or that can allow the dock 200 to secure to a subject (for example, skin of the subject). For example, with reference to FIG. 4E, dock 200 can include one or more of substrates 210, 220, 240, 250, and/or 260.

Substrate 210 can be configured to surround a portion of frame 230. For example, substrate 210 can include an opening 212. Opening 212 can be sized and/or shaped to surround arms 234a, 234b and wall 233. Substrate 210 can be made of foam material such as white polyethylene, polyurethane, or reticulated polyurethane foams, to name a few. Substrate 210 can be made of medical-grade foam material. Substrate 210 can have a perimeter that is greater than a perimeter of rim 231 of frame 230 in some implementations. Substrate 210 and rim 231 can sandwich a substrate 220 therebetween. Substrate 220 can have an opening 222 that is sized and/or shaped to surround arms 234a, 234b and wall 233. Substrate 220 can comprise polyethylene with an adhesive on one or both sides thereof, which can allow substrate 210 to secure to frame 230 (for example, rim 231 of frame 230) in some implementations. Substrate 240 can be positioned between rim 231 and a substrate 250 (described further below). Substrate 240 can include an opening 242 as shown. In some implementations, openings 222 and 242 are identical. Substrate 240 can comprise polyethylene with an adhesive on one or both sides thereof, which can help secure substrate 250 to frame 230 (for example, rim 231 of frame 230). Substrate 240 can be sandwiched between rim 231 and substrate 250.

Substrate 250 can contact and/or secure to skin of a subject when wearable device 100 is in use. Substrate 250 can be a bottommost portion of wearable device 100 when wearable device 100 is in use (for example, after a release liner 260 is removed). Substrate 250 can be or include a material configured to secure to skin of a subject. Substrate 250 can comprise a material configured to allow for removable securement of wearable device 100 to the subject's skin. For example, the substrate 250 can be coated with a high tack, medical-grade adhesive, which when in contact with the subject's skin, is suitable for long-term monitoring, such as, for example two days or longer, such as 3 days, 4 days, 5 days, 6 days, 7 days, 8 days, 9 days, or 10 days or longer. Additionally or alternatively, the substrate 250 can be or include a soft, comfortable, and/or breathable material. For example, substrate 250 can be or include fabric, such as a spunlace fabric. The substrate 250 can include an adhesive material or layer (such as adhesive tape). Substrate 250 can comprise a silicon adhesive with a fabric layer. Such configuration can allow wearable device 100 to comfortably secure to the subject's skin. Substrate 250 can provide thermal insulation and/or provide thermal conductivity. For example, when wearable device 100 is positioned on and/or secured to (for example, adhered to) a subject's skin surface, substrate 250 can act to insulate the skin surface at, around, and/or proximate to a point or region where temperature is measured and/or where thermal energy is transmitted from the skin surface of the subject to or near one or more temperature sensors of wearable device 100 (for example, via thermally conductive probes 344a, 344b). For example, when wearable device 100 is positioned on and/or secured to (for example, adhered to) a subject's skin surface, substrate 250 can insulate the skin surface and can transmit thermal energy to thermally conductive probes 344a, 344b, which in turn, can transmit thermal energy to and/or toward temperature sensors 340a, 340c as described further below.

Substrate 260 can be a release liner 260. Substrate 260 can secure to one or more of the above-described substrates (such as substrate 250) and can be removed prior to securement of wearable device 100 to a subject. For example, substrate 260 can be removed from the substrate 250 prior to placement and/or securement of wearable device 100 on the subject's skin.

FIGS. 5A-5B illustrate top perspective views of hub 300, FIG. 5C illustrates a bottom perspective view of hub 300, and FIG. 5D illustrates a side view of hub 300 which can be a mirror image of the opposite side view of hub 300. Hub 300 can have a first end 302, a second end 304 opposite the first end 302, a first side 306, and a second side 308 opposite the first side 306.

As discussed above, hub 300 can be configured to be removably secured to dock 300, for example, via interaction between recesses 307a, 307b and protrusions 236a, 236b of arms 234a, 234b. Hub 300 can include a housing which can itself include shells 300a, 300c (see FIGS. 5E-5F) which can be secured (for example, permanently secured together) to enclose electronic components of wearable device 100. With reference to FIG. 5A, a portion 302a of end 302 of hub 300 (which can be defined by portions of shells 300a, 300c of a housing of hub 300) can be sized and/or shaped to receive and/or conform to a shape of arm 234a. Similarly, with reference to FIG. 5B, a portion 304a of end 304 of hub 300 (which can be defined by portions of shells 300a, 300c of a housing of hub 300) can be sized and/or shaped to receive and/or conform to a shape of arm 234b. Such portions 302a, 304a can be recessed from an outer surface of housing of hub 300 (for example, formed by shells 300a, 300c).

Recessed portions 302a, 304a can include structure(s) that facilitate engagement and/or securement with protrusions 236a, 236b of arms 234a, 234b of frame 230 of dock 200. For example, portion 302a can include a recessed portion 303a, a recess 307a (which may also be referred to as a “groove”) and a wall 305a (which may also be referred to as a “non-recessed portion”) that at least partially separates recessed portion 303a and recess 307a. Similarly, portion 304a can include a recessed portion 303b, a recess 307b (which may also be referred to as a “groove”) and a wall 305b (which may also be referred to as a “non-recessed portion”) that at least partially separates recessed portion 303b and recess 307b. In some implementations, hub 300 can be secured to dock 200 be inserting hub 300 between arms 234a, 234b of dock 200 from above (see, for example, FIGS. 3A-3B). During such insertion, protrusions 236a, 236b can contact and/or slide along recessed portions 303a, 303b, slide over walls 305a, 305b, and move into recesses 307a, 307b. In some implementations, protrusions 236a, 236b are configured for snap-fit engagement with recesses 307a, 307b (which can be facilitated by recessed portions 303a, 303b and/or walls 305a, 305b). As mentioned above, in some implementations, protrusions 236a, 236b have a beveled or chamfered end which can help protrusions 236a, 236b slide over walls 305a, 305b and into recesses 307a, 307b. Hub 300 and dock 200 can be decoupled from one another by flexing arms 234a, 234b outward, thereby moving protrusions 236a, 236b out of recesses 307a, 307b, as explained in more detail above. In some implementations, hub 300 includes features that can facilitate gripping and/or handling of hub 300, for example, before, during, and/or after removal of hub 300 and dock 200 from one another. For example, hub 300 can include recessed portions 309a, 309b and/or protrusions 311a, 311b extending along a portion of lengths of sides 308, 306.

With reference to FIG. 5C, hub 300 can include a button 325. Button 325 can be utilized to transition hub 300 (and wearable device 100) from a non-operational mode to an operational mode, and vice versa. In some implementations, when hub 300 (and wearable device 100) is in the non-operational mode, electronic functionality of wearable device 100 is disabled, for example, wireless communication is not allowed and/or physiological measurements (such as body temperature) are not determined. Conversely, in some implementations, when hub 300 (and wearable device 100) is in the operational mode, measurements of physiological parameters (such as body temperature) are enabled and/or wireless communication with separate devices is enabled. In some implementations, button 325 is located on a portion of hub 300 such that button 325 is inaccessible when hub 300 and dock 200 are coupled together. For example, with reference to at least FIGS. 1A-1B, 3A-3B, 4A-4B, and 5C, when hub 300 and dock 200 are coupled together, button 325 can face toward substrate 250 and can be hidden. Such configurations can advantageously inhibit or prevent wearable device 100 from being turned off when wearable device 100 is secured to the subject's skin and/or when hub 300 and dock 200 are connected. Such configurations can advantageously prevent a subject from intentionally or unintentionally turning wearable device 100 off when wearable device 100 is secured to the subject (which can ensure proper compliance in some situations).

In some implementations, hub 300 includes an opening 315 configured to allow light from and LED housed within the hub 300 to exit the hub 300 and illuminate nearby areas. This can be utilized to indicate a status of wearable device 100. Opening 315 can be located in shell 300a, which can form a housing when secured to shell 300c.

FIGS. 5E-5F illustrate exploded perspective views of hub 300. Hub 300 can include a housing formed by shell 300a (which may be referred to as “top shell”) and shell 300c (which may be referred to as “bottom shell”). Such housing can enclose electronic components of wearable device 100. Shells 300a, 300c can be permanently secured together. In some implementations, shells 300a, 300c are secured together so as to prevent water ingress into an interior of a housing formed by shells 300a, 300c, which can in turn protect electronic components contained therewithin. With reference to at least FIG. 5T, in some implementations, joining edges represented by numeral “399” in FIG. 5T (for example, a bottom edge of shell 300a and a top edge of shell 300c) can be ultrasonically welded together to prevent water ingress. In some implementations, dock 200 does not include any electronic components and all electronic components of wearable device 100 are contained in hub 300. As shown in FIGS. 5E-5F, wearable device 100 (for example, hub 300) can include an electronics assembly represented by numeral “300b” for purposes of convenience. FIGS. 5G-5H illustrate views of shell 300a and FIGS. 5I-5J illustrate views of shell 300c. FIGS. 5K-5O illustrate views of various electronic and/or structural components that can be enclosed in a housing of hub 300 and can form electronics assembly 300b. Use of the phrase “electronics assembly” and use of numeral “300b” in the present disclosure is not intended to be limiting, but rather, is merely intended as a convenient method to refer to one or more components of wearable device 100 which can be enclosed by the shells 300a, 300c. The use of such phrase and such numeral is not intended to convey that the inclusion of any elements or features described with reference to electronics assembly 300b necessarily requires inclusion of any or all other elements or features described with reference to electronics assembly 300b.

As mentioned above, FIGS. 5G-5H illustrate bottom perspective views of shell 300a. Shell 300a can include various structure(s) that can engage with portions of electronics assembly 300b and/or act to operably position portions of electronics assembly 300b. For example, shell 300a can include structure(s) that engage and/or operably position any or all of circuit boards 330, 331 (see FIGS. 5K-5L). For example, shell 300a can include walls 313a, 313b, 313c (which may also be referred as “arms” or “protrusions”) that can extend through openings 330d in circuit board 330 (see FIGS. 5K-5L, 5O-5Q). Such configurations can help align and/or position circuit board 330 with respect to shell 300a and/or within housing formed by shells 300a, 300c when assembled. Such configurations can also inhibit or limit movement of circuit board 330 in a direction along a plane formed by circuit board 330. With continued reference to FIGS. 5G-5H, shell 300a can include walls 317a, 317b, 316a, 316b, 314a, 314b that can contact a surface of circuit board 331 and provide a platform for circuit board 331. Walls 317a, 317b, 316a, 316b can be received by openings 330b (which can also be referred to as “notches”) located along an edge and/or perimeter of circuit board 330 (see FIGS. 5K-5L). Walls 314a, 314b can at least partially form a cavity 314c that can be sized and/or shaped to receive NFC transponder 333 (see FIGS. 5K-5P). Walls 313a, 313b, 313c, 314a, 314b, 317a, 317b can extend outward from an interior surface 312 of shell 300a as shown. As also shown in FIGS. 5G-5H, shell 300a can include a cavity defined by an enclosure 319 extending outward from surface 312 of shell 300a and which can be positioned around emitter 397 (see FIG. 5K-5L). Opening 315 can extend through a portion of shell 300a into such cavity defined by enclosure 319, as shown. Enclosure 319 can isolate emitter 397 and/or light emitted from emitter 397 and help guide emitted light through opening 315.

FIGS. 5I-5J illustrate bottom perspective views of shell 300c. Shell 300c can include various structure(s) that can engage with portions of electronics assembly 300b and/or act to operably position portions of electronics assembly 300b. For example, shell 300c can include structure(s) that engage and/or operably position any or all of circuit boards 330, 331 (see FIGS. 5K-5L). For example, shell 300c can include arms 321a, 321b, 321c, 321d that extend outward from (for example, generally perpendicular to) an interior surface 320 of shell 300c. Arms 321a, 321b, 321c, 321d can be configured to engage with notches and/or openings in circuit boards 330, 331. For example, circuit board 330 can include openings 330c located in an interior portion of circuit board 300 and openings 330a (which can also be referred to as “notches”) located along an edge and/or perimeter of circuit board 330 (see FIGS. 5K-5L, 5P, 5Q). FIG. 5P illustrates shell 300c coupled with electronics assembly 300b without also showing shell 300a, frame 334, and antenna 335 for purposes of clarity. As shown, portions of arms 321a, 321b, 321c, 321d can be received in openings 330a and ends of arms 321a, 321b, 321c, 321d can extends through openings 330c. In some implementations, arms 321a, 321b, 321c, 321d include a notch near free ends thereof, and such notch is configured to receive (for example, “grip”) portions of circuit board 330 near openings 330c, 330a. As shown in at least FIGS. 5K-5L, circuit board 331 can include openings 331a (which can also be referred to as “notches”) located along an edge and/or perimeter of circuit board 331, and portions of arms 321a, 321b, 321c, 321d can be received in openings 331a. Such configurations can advantageously allow shell 300c to engage and/or operably position circuit boards 330, 331, for example, within housing defined by shells 300a, 300c when assembled. With reference to FIGS. 5I-5J, in some implementations, free ends of arms 321a, 321b, 321c, 321d are chamfered or beveled, which can help such free ends contact and slide into engagement with openings 330c.

As discussed above, wearable device 100 can include a button 325 that can transition wearable device 100 between operational and non-operational modes. In some implementations, button 325 can be defined by a portion of shell 300c that can be recessed from interior surface 320 of shell 300c (see FIGS. 5I-5J). Button 325 can include an actuator portion and a switch. Such actuator portion can include a pad 325a (which can be pressable by a user, for example, by a finger of a user) and a protrusion 325b that can extend outward from pad 325a (which can have a cross shape among others). Protrusion 325b can be configured to engage a switch 325c (see FIGS. 5M-5N) coupled to circuit board 331. Movement (for example, actuation) of pad 325a can move protrusion 325b into engagement with switch 325c, which can cause wearable device 100 to transition between operational and non-operational modes. Button 325 can be an embodiment of user input device 108 discussed above.

With continued reference to FIGS. 5I-5J, shell 300c can include a wall 322 that extends between arms 321c, 321d adjacent to button 325 and/or can extend around a portion of a perimeter of button 325. Wall 322 can be curved and/or partially curved to correspond to a curvature of battery 332 (see FIGS. 5K-5O). Wall 322 can act to operably position battery 332.

As shown in at least FIGS. 5I-5J, shell 300c can include openings 324a, 324b, which can be positioned within an interior of shell 300c and/or spaced from a perimeter of shell 300c. Openings 324a, 324b can allow thermally conductive probes 344a, 344b to extend through shell 300c and a housing formed by shells 300a, 300c. Such configuration can allow probes 344a, 344b to contact substrate 250 when hub 300 and dock 200 are secured together, which can in turn allow probes 344a, 344b to receive thermal energy from substrate 250 (from skin of the user) and transmit such thermal energy to and/or toward temperature sensors 340a, 340c as described further below.

FIGS. 5K-5L illustrate top perspective views of electronics assembly 300b of hub 300 and FIGS. 5M-5N illustrate bottom perspective views of electronics assembly 300b. FIG. 5O illustrate a partially exploded bottom perspective view of electronics assembly 300b. FIGS. 5Q-5R illustrate top views of circuit boards 330, 331 (respectively). FIG. 5S illustrates a cross-section taken through electronics assembly 300b shown in FIGS. 5E-5F.

Wearable device 100 can include circuit boards 330, 331 as discussed above. Circuit boards 330, 331 can mechanically support and electrically connect various electrical components of wearable device 100 to facilitate the performance of various functions of wearable device 100. Such electrical components can include without limitation, processor 101, storage device 102, communication module 103, and one or more temperature sensors 106 (which can be, for example, temperature sensors 340a, 340b, 340c, 340d), one or more other sensor(s) 107, and/or other components discussed elsewhere herein. Circuit boards 330, 331 can be spaced apart from one another by a gap. In some implementations, circuit boards 330, 331 are oriented substantially parallel to one another. As shown, battery 332 can be positioned between circuit boards 330, 331. In some implementations, circuit boards 330, 331 are mechanically and/or electrically coupled with one or more headers 336, which can facilitate communication between circuit boards 330, 331 (for example, signals communicated therebetween). Such headers 336 can act to maintain the spacing and/or orientation of circuit boards 330, 331 with respect to each other.

Battery 332 can be an embodiment of battery 104 discussed above. Battery 332 can provide power to the hardware components of wearable device 100 which are described herein. Battery 332 can be a coin cell battery (such as a lithium coin cell battery). Battery 332 can have a circular shape. Battery 332 can comprise a metal housing. Battery 332 can be in electrical contact with circuit board 330 and/or circuit board 331. For example, as discussed below, battery 332 can contact an electrical contact 357 on circuit board 331. In some implementations, battery 332 is not rechargeable. With reference to FIGS. 5O and 5S, wearable device 100 can include an electrical contact 349 coupled to a surface (for example, bottom surface) of circuit board 330 and configured to contact a portion of battery 332. In some implementations, electrical contact 349 has a spring-like configuration which applies a compressive force against a portion of battery 332 when battery 332 is assembled between circuit boards 330, 331.

Wearable device 100 can include near field communication (NFC) functional capabilities (for example, RFID) that can enable wearable device 100 to interact and/or communicate with separate computing devices. Such NFC functional capabilities can enable wearable device 100 to, among other things: confirm or verify that it is and/or is made up of authentic components; transfer data (for example, physiological data) obtained by wearable device 100; and determine a lifespan of the wearable device 100. For example, wearable device 100 can include NFC transponder 333 (for example, in the form of a chip) that can interact with an RFID reader of a separate computing device that emits a radio frequency. NFC transponder 333 can be an embodiment of and/or be part of communication module 103 discussed above. NFC transponder 333 can be positioned within housing of hub 300 defined by shells 300a, 300c. NFC transponder 333 can be positioned near an exterior portion of the housing, for example, within cavity 314c discussed above.

Wearable device 100 can include an antenna 335 to facilitate wireless communication. Antenna 335 can be an embodiment of and/or be part of communication module 103 discussed above. Antenna 335 can allow wearable device 100 to wirelessly communicate via any of the communication protocols discussed elsewhere herein, such as but not limited to, Wi-Fi (802.11x), Bluetooth®, ZigBee®, Z-wave®, cellular telephony, infrared, near-field communications (NFC), RFID, satellite transmission, proprietary protocols, combinations of the same, and the like. As discussed previously and as shown in the figures, wearable device 100 can include a battery 332, which, in some implementations, is placed between and/or adjacent circuit boards 330, 331. In some cases, the battery 332 comprises a metal housing that can negatively impact antenna range. Advantageously, in some implementations where battery 332 is positioned adjacent circuit boards 330, 331, antenna 335 is positioned spaced away from the circuit boards 330, 331 in order to minimize the affect of the battery 332 on the range of the antenna 335. Such configurations can also position the antenna 335 further away from the user's skin and body when wearable device 100 is secured to the user, which can also improve antenna range since the body can negatively impact antenna range. For example, in some implementations, wearable device 100 include a frame 334 coupled to circuit board 330 and/or 331 that mounts the antenna 335 and positions the antenna 335 away from circuit boards 330, 331. Antenna 335 can be positioned and/or secured atop a top surface of frame 334, as shown. Numerals 335a, 335b in FIGS. 5K-5L are used to indicate where portions of antenna 335 are connected to (for example, soldered) circuit board 330. As shown, end portions of antenna 335 (at/near numerals 335a, 335b) can be connected to circuit board 330 and can extend upward over ends of legs 334c, 334d of frame 334 to allow a remainder of antenna 335 to be positioned atop the top surface of frame 334 as shown.

In some implementations, frame 334 comprises one or more legs 334a, 334b, 334c, 334d (see FIG. 5K-5L). Legs 334b, 334c, 334d can extend transverse (for example, generally perpendicular) to leg 334a. Frame 334 can include structure(s) that engage with structure(s) of circuit board 330. For example, as shown in FIG. 5N, frame 334 can include protrusions 334e extending outward from leg 334a (for example, generally perpendicular to leg 334) which can be received within openings 330e (which may be referred to as “notches”) located along an edge and/or perimeter of circuit board 330 (see FIG. 5Q). Additionally or alternatively, frame 334 can include legs 334d which can extend from leg 334a. Legs 334d can extend from leg 334a in a first direction (for example, generally perpendicular to leg 334a, which is shown as “downward” in FIG. 5N) and in a second direction that is generally perpendicular to the first direction and which can be substantially parallel to legs 334b, 334c, 334d. Legs 334d can engage with openings 330f (which may be referred to as “notches”) located along an edge and/or perimeter of circuit board 330 (see FIG. 5Q).

Wearable device 100 can include one or more indicators configured to indicate a status of wearable device 100, such as whether wearable device 100 is in an operational (“on”) mode, whether wearable device 100 is pairing or has paired with a separate device, whether an error has been detected, and/or a power level of wearable device 100. For example, with reference to at least FIGS. 5K-5L, the wearable device 100 can include an emitter 397 configured to emit light of one or more wavelengths to indicate a status of wearable device 100. The emitter 397 can be coupled to the circuit board 330. The emitter 397 can include one or more light-emitting diodes (LEDs). The emitter 397 can emit light of certain colors to indicate certain statuses of wearable device 100. For example, the emitter 397 can emit a green light to indicate that wearable device 100 is powered “on” or a red light to indicate wearable device 100 is “off”. A housing formed by shells 300a, 300c can include an opening configured to allow light emitted from the emitter 397 to be visible from a location outside an interior of the housing. For example, as discussed above, shell 300a can include opening 315. Additionally or alternatively, shells 300a and/or 300c can comprise a transparent or semi-transparent material that allows light emitted from the emitter 397 to be seen from a location outside an interior of the housing. In some implementations, opening 315 is at least partially aligned with emitter 397 to allow light emitted from the emitter 397 to more easily pass through the housing.

FIGS. 5Q and 5R illustrate top views of circuit boards 330, 331. As discussed herein, wearable device 100 can include one or more temperature sensors that can be mounted to circuit boards 330, 331. For example, wearable device 100 can include temperature sensors 340b and 340d mounted to circuit board 330 and temperature sensors 340a and 340c mounted to circuit board 331. In some implementations, circuit board 330 can include one or more openings at least partially between temperature sensors 340b and 340d and/or circuit board 331 can include one or more openings at least partially between temperature sensors 340a, 340c. Such one or more openings can be positioned within an interior portion of circuit boards 330, 331. Such one or more openings in circuit board 330 can be positioned such that a line extending between temperature sensors 340b and 340d passes through at least a portion of the one or more openings. Similarly, such one or more openings in circuit board 331 can be positioned such that a line extending between temperature sensors 340a and 340c passes through at least a portion of the one or more openings.

For example, circuit board 330 can include an opening 330g and/or an opening 330h. Openings 330g, 330h can be positioned at least partially between temperature sensors 340b, 340d such that a line extending between temperature sensors 340b and 340d passes through at least a portion of openings 330g, 330h. Opening 330g can be positioned proximate temperature sensor 340b and/or opening 330h can be positioned proximate temperature sensor 340d. Similarly, circuit board 331 can include an opening 331c and/or an opening 331d. Openings 331c, 331d can be positioned at least partially between temperature sensors 340a, 340c such that a line extending between temperature sensors 340a and 340c passes through at least a portion of openings 331c, 331d. Opening 331c can be positioned proximate temperature sensor 340a and/or opening 331d can be positioned proximate temperature sensor 340c. Openings 330g, 330h, 331c, 331d can be defined by one or more linear portions. For example, opening 330g can be defined by two linear portions which are transverse (for example, generally perpendicular) to one another. As another example, opening 330h can be defined by a single linear portion. In some implementations, openings 331c, 331d are defined by three linear portions, two of which are generally parallel to one another and one of which is generally perpendicular to the other two.

Advantageously, openings 330g, 330h can inhibit or minimize heat flow along circuit board 330 between temperature sensors 340b and 340d, and openings 331c, 331d can inhibit or minimize heat flow along circuit board 331 between temperature sensors 340a and 340c. Such configurations can allow temperature sensors 340a, 340b, 340c, 340d to be utilized to capture unique temperature values which can be advantageous in determining internal body temperature as explained in more detail below.

With reference to FIG. 5R, wearable device 100 can include an electrical contact 357 on circuit board 331 that can be electrically coupled to battery 332 when assembled. In some implementations such as that shown, electrical contact 357 comprises a circular shape corresponding to a shape of battery 332. Additionally or alternatively, in some implementations, electrical contact 357 can comprise a hatched pattern. Such hatching can inhibit or minimize heat flow along circuit board 330 between temperature sensors 340b and 340d. Additionally or alternatively, such hatching can inhibit or minimize heat flow along circuit board 331 between thermally conductive probes 344a, 344b which can be in contact with circuit board 331. Advantageously, such hatching can thereby allow temperature sensors 340a, 340b, 340c, 340d to be utilized to capture unique temperature values which can be advantageous in determining internal body temperature as explained in more detail below. Electrical contact 357 can comprise copper, for example, electrical contact 357 can be a gold plated copper pad.

With reference to FIGS. 5M-5O and as discussed elsewhere herein, wearable device 100 can include thermally conductive probes 344a, 344b. Thermally conductive probes 344a, 344b can advantageously help transmit thermal energy to and/or toward temperature sensors 340a, 340c, as also discussed elsewhere herein. Thermally conductive probes 344a can be rigid. Thermally conductive probes 344a, 344b can comprise a metallic material. In some implementations, probes 344a, 344b comprise brass. In some implementations, probes 344a, 344b are made of the same material. In some implementations, probes 344a, 344b are made of a different material. Thermally conductive probes 344a can comprise a circular cross-section. Thermally conductive probes 344a can have a first end that is substantially flat and a second end that is tapered (see FIG. 5O).

Thermally conductive probe 344a can have a first end positioned adjacent and/or secured to a portion of circuit board 331 and a second end opposite such first end. In some implementations, thermally conductive probe 344a (for example, such first end thereof) is soldered to circuit board 331. Such first end of thermally conductive probe 344a can be positioned adjacent and/or secured to a portion of circuit board 331 such that probe 344a is substantially aligned with one or both of temperature sensors 340a, 340b (see FIGS. 5K and 5S). Thermally conductive probe 344b can have a first end positioned adjacent and/or secured to a portion of circuit board 331 and a second end opposite such first end. In some implementations, thermally conductive probe 344b (for example, such first end thereof) is soldered to circuit board 331. Such first end of thermally conductive probe 344b can be positioned adjacent and/or secured to a portion of circuit board 331 such that probe 344b is substantially aligned with one or both of temperature sensors 340c, 340d (see FIGS. 5L and 5S).

Circuit board 331 can include one or more openings configured to allow thermal energy from thermally conductive probes 344a, 344b to pass through circuit board 331 and to temperature sensors 340a, 340c. For example, as shown in FIG. 5O, circuit board 331 can include a first plurality of holes 348a and a second plurality of holes 348b. Holes 348a can be arranged in an array and/or group proximate one another and can be positioned between thermally conductive probe 344a and temperature sensor 340a. Similarly, holes 348b can be arranged in an array and/or group proximate one another and can be positioned between thermally conductive probe 344b and temperature sensor 340c. The plurality of holes 348a can comprise two, three, four, five, six, seven, or eight or more holes 348a. Alternatively, such plurality of holes 348a could be replaced with a single hole. The plurality of holes 348b can comprise two, three, four, five, six, seven, or eight or more holes 348b. Alternatively, such plurality of holes 348b could be replaced with a single hole. In some implementations, such holes 348a, 348b are filled with a thermally conductive material. In some implementations, such holes 348a, 348b are not filled with a thermally conductive material. In some implementations, such holes 348a, 348b are left void. In some implementations, holes 348a are filled with a thermally conductive material but holes 348b are not filled with a thermally conductive material, or holes 348b are filled with a thermally conductive material but holes 348a are not filled with a thermally conductive material.

Thermally conductive probes 344a, 344b can be configured to contact substrate 250 when wearable device 100 is in use (for example, when hub 300 and dock 200 are coupled together. A housing defined by shells 300a, 300c can include openings configured to allow probes 344a, 344b to extend through the housing and contact substrate 250 when wearable device 100 is in use. For example, shell 300c can include openings 324a, 324b that can be sized and/or shaped to allow probes 344a, 344b to extend therethrough.

In some implementations, wearable device 100 includes seals configured to prevent water ingress through openings 324a, 324b around probes 344a, 344b. For example, wearable device 100 can include O-rings 345a, 345b that can surround probes 344 a, 344 b and sit within recessed portions of shell 300c surrounding openings 324a, 324b (see FIGS. 5I-5J and 5T).

Circuit board 330 can include one or more openings configured to allow thermal energy to pass through circuit board 330 and to temperature sensors 340b, 340d. For example, with reference to FIG. 5O, circuit board 330 can include a first plurality of holes 350a and a second plurality of holes 350b. Holes 350a can be arranged in an array and/or group proximate one another and can be positioned adjacent to (for example, underneath) temperature sensor 340b. Similarly, holes 350b can be arranged in an array and/or group proximate one another and can be positioned adjacent to (for example, underneath) temperature sensor 340d. The plurality of holes 350a can comprise two, three, four, five, six, seven, or eight or more holes 350a. Alternatively, such plurality of holes 350a could be replaced with a single hole. The plurality of holes 350b can comprise two, three, four, five, six, seven, or eight or more holes 350b. Alternatively, such plurality of holes 350b could be replaced with a single hole. In some implementations, such holes 350a, 350b are filled with a thermally conductive material. In some implementations, such holes 350a, 350b are not filled with a thermally conductive material. In some implementations, such holes 350a, 350b are left void. In some implementations, holes 350a are filled with a thermally conductive material but holes 350b are not filled with a thermally conductive material, or holes 350b are filled with a thermally conductive material but holes 350a are not filled with a thermally conductive material.

With reference to FIG. 5S, wearable device 100 can include temperature sensors 340a, 340b, 340c, 340d. Temperature sensors 340a, 340c can be mounted to a first surface of circuit board 331 and spaced away from each other. Thermally conductive probes 344a, 344b can be positioned adjacent and/or secured to a second surface of circuit board 331 that is opposite the first surface of circuit board 331 that temperature sensors 340a, 340c are mounted to. Holes 348a can be positioned at least partially between temperature sensor 340a and probe 344a and holes 348b can be positioned at least partially between temperature sensor 340c and probe 344b.

Temperature sensors 340a, 340b, 340c, 340d can be configured to generate one or more signals responsive to detected thermal energy, determine temperature, and/or transmit such generated one or more signals and/or such determined temperature to the processor 101 of wearable device 100 continuously and/or intermittently. For example, temperature sensors 340a, 340b, 340c, 340d can be configured to generate one or more signals responsive to detected thermal energy, determine temperature, and/or transmit such generated one or more signals and/or such determined temperature every 0.5 seconds, 1 second, 2 second, 3 seconds, 4 seconds, 5 seconds, 10 seconds, 30 seconds, 1 minute, 2 minute, 3 minutes, 4 minutes, 5 minutes, or at other intervals. Such generated one or more signals, determined temperature, and/or transmission of such generated one or more signals and/or determined temperature for each of temperature sensors 340a, 340b, 340c, 340d can be simultaneous or non-simultaneous.

The various devices, methods, and/or systems discussed above can be used for monitoring a subject's physiological information. For example, as discussed above, wearable device 100 can be used to measure a subject's temperature over time. As discussed above, wearable device 100 can be configured to wirelessly communicate with a separate computing device, such as a patient monitor and/or a mobile device (e.g., smart phone). Wearable device 100 can wirelessly transmit physiological data (such as temperature data) over time (continuously or periodically) to such separate computing device for display, among other things. As also discussed above, wearable device 100 can wirelessly transmit processed or unprocessed obtained physiological information to a mobile phone (for example) which can include one or more hardware processors configured to execute an application that generates a graphical user interface displaying information representative of the processed or unprocessed physiological information obtained from wearable device 100. Such graphical user interfaces can display continuous and/or periodic measurements obtained from wearable device 100, display and/or issue various types of alerts, display physiological trend information (for example, temperature trends), among other things. Features or aspects displayed by such graphical user interfaces can include, without limitation, a splash screen, onboarding, device setup, instructions (for example, both visual/graphical and textual) for securing wearable device 100 to a subject and/or pairing wearable device 100 to the separate computing device, temperature data and/or trending dashboard, user scenarios, notes (such as medication notes and reminders as well as other user activity notes), temperature trending data and information, user settings and profiles, app settings, and/or alerts and push notifications.

Temperature sensors 340b, 340d can be mounted to a first surface of circuit board 330 and spaced away from each other. A second surface of circuit board 330 that is opposite the first surface of the circuit board 331 can face toward temperature sensors 340a, 340c and toward circuit board 331 (for example, toward the first surface of circuit board 331 that temperature sensors 340a, 340c are mounted to). Circuit board 330 (for example, second surface of circuit board 330 that is opposite the first surface of circuit board 330 that temperature sensor 340b, 340d are mounted to) can be spaced a distance di from temperature sensor 340a. Such distance di can be approximately 0.5 mm, approximately 1 mm, approximately 1.5 mm, approximately 2 mm, approximately 2.5 mm, approximately 3 mm, approximately 3.5 mm, or approximately 4 mm, or any value or range between any of these values, or any value or range bounded by any combination of these values. Circuit board 330 (for example, second surface of circuit board 330 that is opposite the first surface of circuit board 330 that temperature sensor 340b, 340d are mounted to) can be spaced a distance d2 from temperature sensor 340 c. Such distance d2 can be approximately 0.5 mm, approximately 1 mm, approximately 1.5 mm, approximately 2 mm, approximately 2.5 mm, approximately 3 mm, approximately 3.5 mm, or approximately 4 mm, or any value or range between any of these values, or any value or range bounded by any combination of these values. Distance d1 and/or d2 can be at least approximately 0.1 mm, at least approximately 0.2 mm, at least approximately 0.3 mm, at least approximately 0.4 mm, at least approximately 0.5 mm, at least approximately 1 mm, at least approximately 1.5 mm, at least approximately 2 mm, at least approximately 2.5 mm, at least approximately 3 mm, at least approximately 3.5 mm, or at least approximately 4 mm.

With continued reference to FIG. 5S, temperature sensor 340b, temperature sensor 340a, and thermally conductive probe 344a can be substantially aligned with one another along and/or with respect to an axis 5 that is parallel to axis 1 (see FIG. 5S and FIGS. 5K-5L). An axis extending through a center of an array or group of holes 350a and/or holes 348a can substantially align with such axis 5 and/or be substantially parallel to axis 1. Similarly, temperature sensor 340d, temperature sensor 340c, and thermally conductive probe 344b can be substantially aligned with one another along and/or with respect to an axis 7 that is parallel to axis 1 (see FIG. 5S and FIGS. 5K-5L). An axis extending through a center of an array or group of holes 350b and/or holes 348b can substantially align with such axis 5 and/or be substantially parallel to axis 1. As shown, axis 5 and 7 can be spaced from one another, for example, in a direction along axis 2. Each of axes 1, 2, and 3 are shown in FIGS. 5K, 5L, and 5S, and are mutually orthogonal to one another.

Temperature sensors 340a, 340b can be thermally insulated from one another. For example, with reference to at least FIG. 5S, an air gap can be present at least partially between temperature sensor 340a and 340b. For example, an air gap can be positioned at least partially between temperature sensor 340a, a second/bottom surface of circuit board 330, holes 350a, and temperature sensor 340b. In some variants, a thermally insulative material is positioned in place of such air gap.

Temperature sensors 340c, 340d can be thermally coupled to one another, for example, by a thermally conductive element 342. Thermally conductive element 342 can be positioned at least partially between temperature sensors 340c, 340d. For example, thermally conductive element 342 can be positioned between temperature sensor 340c and a second surface of circuit board 330 that is opposite a first surface of circuit board 330 to which temperature sensor 340d is mounted. Thermally conductive element 342 can also be positioned between holes 350b and temperature sensor 340c. Thermally conductive element 342 can comprise a first end 342a secured to the second surface of circuit board 330 adjacent to holes 350b, a second end 342c secured to the temperature sensor 340c, and a stem 342b positioned in between the first and second ends 342a, 342c. Thermally conductive element 342 can be rigid. Thermally conductive element 342 can be in a flexed configuration where stem 342b is at least partially bent when assembled (see FIGS. 5L-5M and 5O). Thermally conductive element 342 can comprise a metallic material, such as copper. As another example, the thermally conductive element 342 can comprise beryllium copper (BeCu). In some variants, a thermal material (such as a thermal paste) is positioned between end 342c and temperature sensor 340c, which can advantageously increase thermal transmissivity in some cases. For example, in some variants, such thermal paste comprises zinc oxide and/or is silicone free.

With reference to FIG. 5S, thermally conductive element 342 can have a width (for example, a width of end 342a, end 342c, and/or stem 342b which can extend in a direction parallel to axis 2 as shown in FIG. 5S) that is between approximately 0.01 inch and approximately 1 inch, for example, between approximately 0.01 inch and approximately 0.5 inch, between approximately 0.01 inch and approximately 0.4 inch, between approximately 0.01 inch and approximately 0.3 inch, between approximately 0.01 inch and approximately 0.2 inch, between approximately 0.01 inch and approximately 0.1 inch, between approximately 0.02 inch and approximately 0.1 inch, between approximately 0.03 inch and approximately 0.1 inch, between approximately 0.03 inch and approximately 0.1 inch, between approximately 0.04 inch and approximately 0.1 inch, between approximately 0.05 inch and approximately 0.1 inch, between approximately 0.06 inch and approximately 0.1 inch, between approximately 0.07 inch and approximately 0.1 inch, or between approximately 0.08 inch and approximately 0.1 inch, or any value or range between any of these values, or any value or range bounded by any combination of these values.

A width of stem 342b (see FIG. 5O) of thermally conductive element 342 (extending in a direction parallel to axis 2 as shown in FIG. 5S) can be between approximately 0.01 inch and approximately 1 inch, for example, between approximately 0.01 inch and approximately 0.5 inch, between approximately 0.01 inch and approximately 0.4 inch, between approximately 0.01 inch and approximately 0.3 inch, between approximately 0.01 inch and approximately 0.2 inch, between approximately 0.01 inch and approximately 0.1 inch, between approximately 0.01 inch and approximately 0.1 inch, between approximately 0.01 inch and approximately 0.09 inch, between approximately 0.01 inch and approximately 0.08 inch, between approximately 0.01 inch and approximately 0.07 inch, between approximately 0.01 inch and approximately 0.06 inch, between approximately 0.04 inch and approximately 0.06 inch, or any value or range between any of these values, or any value or range bounded by any combination of these values.

A thickness of thermally conductive element 342 (for example, a thickness of end 342 a, end 342 c, and/or stem 342 b) can be between approximately 0.001 inch and approximately 0.1 inch, for example, between approximately 0.001 inch and approximately 0.09 inch, between approximately 0.001 inch and approximately 0.08 inch, between approximately 0.001 inch and approximately 0.07 inch, between approximately 0.001 inch and approximately 0.06 inch, between approximately 0.001 inch and approximately 0.05 inch, between approximately 0.001 inch and approximately 0.04 inch, between approximately 0.001 inch and approximately 0.03 inch, between approximately 0.001 inch and approximately 0.02 inch, between approximately 0.001 inch and approximately 0.01 inch, between approximately 0.001 inch and approximately 0.009 inch, between approximately 0.001 inch and approximately 0.008 inch, between approximately 0.001 inch and approximately 0.007 inch, between approximately 0.001 inch and approximately 0.006 inch, between approximately 0.004 inch and approximately 0.006 inch, or any value or range between any of these values, or any value or range bounded by any combination of these values.

FIG. 5T illustrates a cross-section taken through wearable device 100 when wearable device 100 is secured to user's skin. As discussed above, it is often difficult to accurately estimate internal body temperature based on temperature measurements obtained via skin. Advantageously, the arrangement of temperature sensors 340a, 340b, 340c, 340d along with various other components of wearable devices disclosed herein can facilitate more robust determinations of internal body temperature.

As discussed previously, wearable device 100 can include a substrate 250 that can be positioned to contact and/or secure to skin of a user when wearable device 100 is in use. Wearable device 100 can be secured to the skin via securement of dock 200 (and substrate 250) to the skin, prior to, during, and/or after securement of hub 300 to dock 200 which is described elsewhere herein. As also described previously, thermally conductive probes 344a, 344b can extend through opening 232 in frame 230 of dock 200 and contact substrate 250 (for example, an interior surface of substrate 250 that is opposite to an exterior or skin-facing surface of substrate 250) when hub 300 and dock 200 are coupled together. In some cases, probes 344a, 344b cause substrate 250 to “bulge”, as shown in FIG. 5T (which may be an exaggerated representation of such “bulging”), for example, due to the length of probes 344a, 344b in relation to dimensions of the hub 300 and/or dock 200 (and/or portions thereof). Such bulging may cause corresponding pressure and/or “bulging” of a portion of the user's skin underneath (see FIG. 5T). In some implementations, the probes 344a, 344b are not configured to cause such “bulging”, but merely contact substrate 250. In some implementations such as that shown, none of temperature sensors 340a, 340b, 340c, 340d and none of thermally conductive probes 344a, 344b contact skin of the user when wearable device 100 is in use. As also shown, when wearable device 100 is in use, probes 344a, 344b can receive thermal energy via contact with substrate 250 which itself contacts and receives thermal energy from skin. Such configurations can provide more consistent temperature readings since moisture and/or other characteristics of skin (for example, oil or dirt levels on skin) may result in inconsistent temperature readings.

Thermal energy radiating from the internal body of the user passing through the skin is conducted through substrate 250 and through thermally conductive probes 344a, 344b. As described previously, thermally conductive probes 344a, 344b can act as a thermal conduit to transmit thermal energy toward temperature sensors 340a, 340c. As discussed above, circuit board 331 can include holes 348a, 348b that can allow such thermal energy to pass through circuit board 331 to temperature sensors 340a, 340c.

In addition to temperature sensors 340a, 340c, wearable device 100 can include temperature sensors 340b, 340d. Temperature sensors 340b, 340d are operably positioned within a housing defined by shells 300a, 300c to be positioned farther away from the user's skin than temperature sensors 340a, 340c when wearable device 100 is secured to the user. For example, as shown, temperature sensors 340b, 340d can be positioned on a surface of circuit board 330 that faces toward a top interior surface of shell 300a. Such arrangement allows temperature sensors 340b, 340d to be more responsive to ambient temperature (for example, environmental temperature outside the housing of wearable device 100). In some variants, thermal putty (for example, a ceramic filled silicone sheet) is positioned between temperature sensors 340b, 340d and the top interior surface of shell 300a in order to provide better thermal contact between temperature sensors 340b, 340d and the ambient environment.

As discussed above, an air gap can be positioned between temperature sensor 340a and temperature sensor 340b (for example, between circuit board 330 and temperature sensor 340a). As also discussed above, a thermally conductive element 342 can be positioned between temperature sensors 340c, 340d. In such configurations, two unique temperature gradients are established, one between temperature sensors 340a, 340b and one between temperature sensors 340c, 340d.

Temperature data from each of temperature sensors 340a, 340b, 340c, 340d can advantageously be utilized by wearable device 100 to facilitate more robust approximations of internal body temperature. For example, temperature data from temperature sensors 340a and 340b can be compared (for example, differences can be determined therebetween) and/or temperature sensors 340c and 340d can be compared (for example, differences can be determined therebetween). Additionally or alternatively, comparisons between temperature data from temperature sensors 340a, 340c and/or between temperature sensors 340b, 340d can be made (for example, differences therebetween can be determined). Additionally or alternatively, comparisons between temperature data from temperature sensors 340a, 340d and/or between temperature sensors 340b, 340c can be made (for example, differences therebetween can be determined). Additionally, known information relating to thermal properties of air (which can be present between temperature sensors 340a, 340b as discussed previously) and/or thermally conductive element 342 can be utilized along with temperature data and/or comparisons of temperature data from temperature sensors 340a, 340b, 340c, 340d to determine robust approximations of internal body temperature. Such information can advantageously be utilized to overcome challenges of estimating internal body temperature based on skin temperature readings which are discussed above. Wearable device 100 (for example, processor 101) can determine body temperature values based on any of such above-described comparisons and/or differences and/or other information. In some implementations, wearable device 100 (for example, processor 101) can determine body temperature values based on one or more comparisons and/or one or more differences between any of such above-described differences.

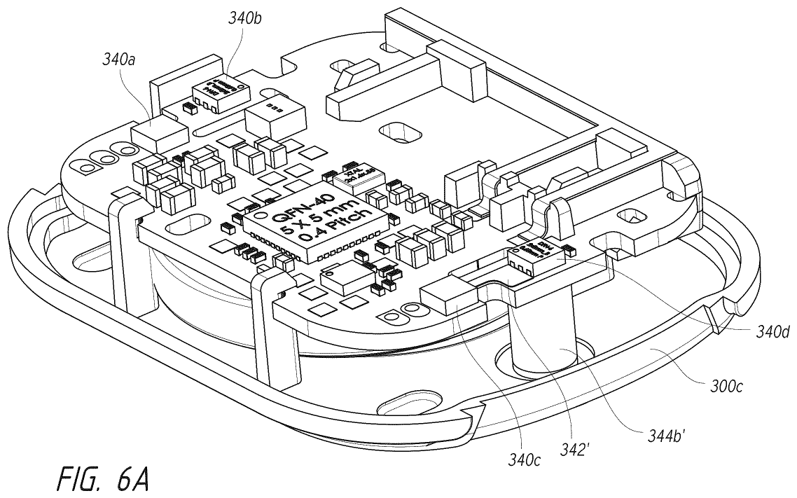

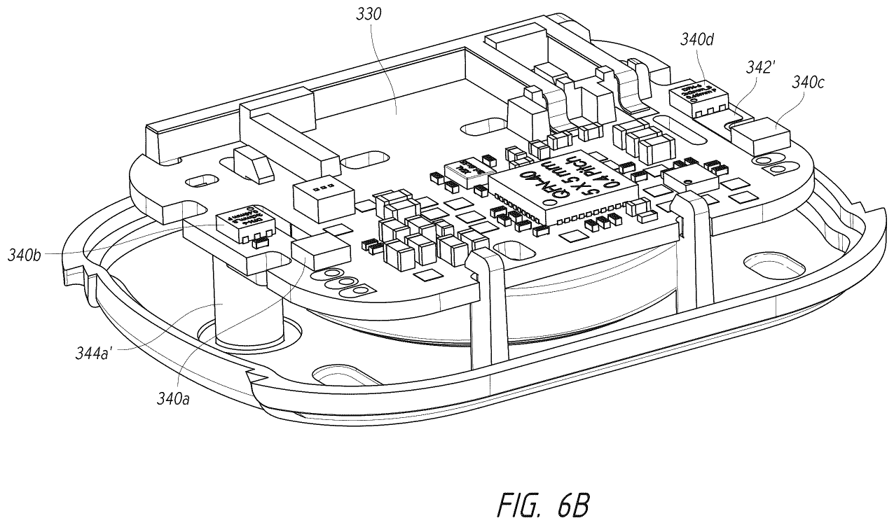

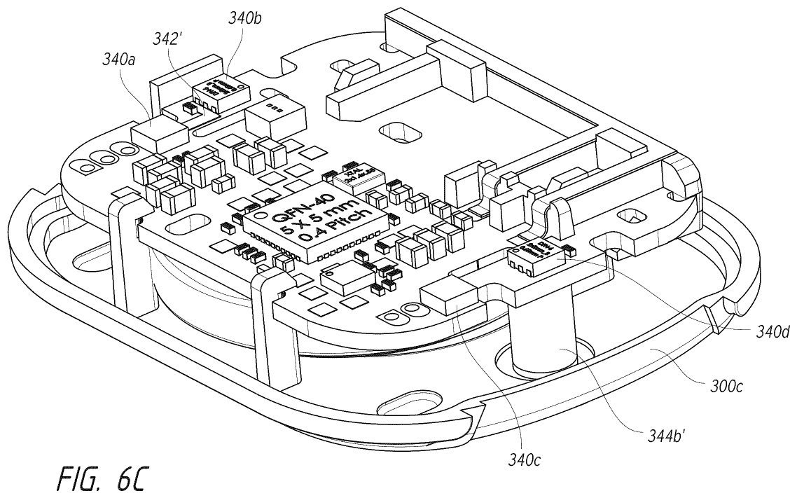

FIGS. 6A-6B illustrate an alternative implementation of hub 300, which includes only one circuit board (for example, circuit board 330), without shell 300a for purposes of clarity. In such alternative implementation, hub 300 includes thermally conductive probes 344a', 344b′ which are longer than probes 344a, 344b and which extend up to a bottom surface of circuit board 330, for example, adjacent holes 350a, 350b (see FIG. 5S). Such alternative implementation still includes temperature sensors 340a, 340b, 340c, 340d, but all are arranged along substantially the same plane on the same surface of circuit board 330, as shown. As shown in FIG. 6A, temperature sensor 340d is substantially aligned with probe 344b′ and temperature sensor 340c is not substantially aligned with probe 344b′ and is spaced from temperature sensor 340d. Similarly, as shown in FIG. 6B, temperature sensor 340b is substantially aligned with probe 344a′ and temperature sensor 340a is not substantially aligned with probe 344a′ and is spaced from temperature sensor 340b. In some implementations, a thermally conductive element 342′ (which can be similar to thermally conductive element 342 in many respects) is connected between temperature sensors 340d, 340c. In a variant as shown in FIG. 6C, thermally conductive element 342′is instead placed between temperature sensors 340a, 340b. Comparisons between different ones of temperature sensors 340a, 340b, 340c, 340d (such as any of the comparisons and/or differences discussed above) can be utilized along with known information relating to thermal properties of air (which can be present between temperature sensors 340a, 340b and/or 340c, 340d) and/or thermally conductive element 342′can be utilized to determine robust approximations of internal body temperature.

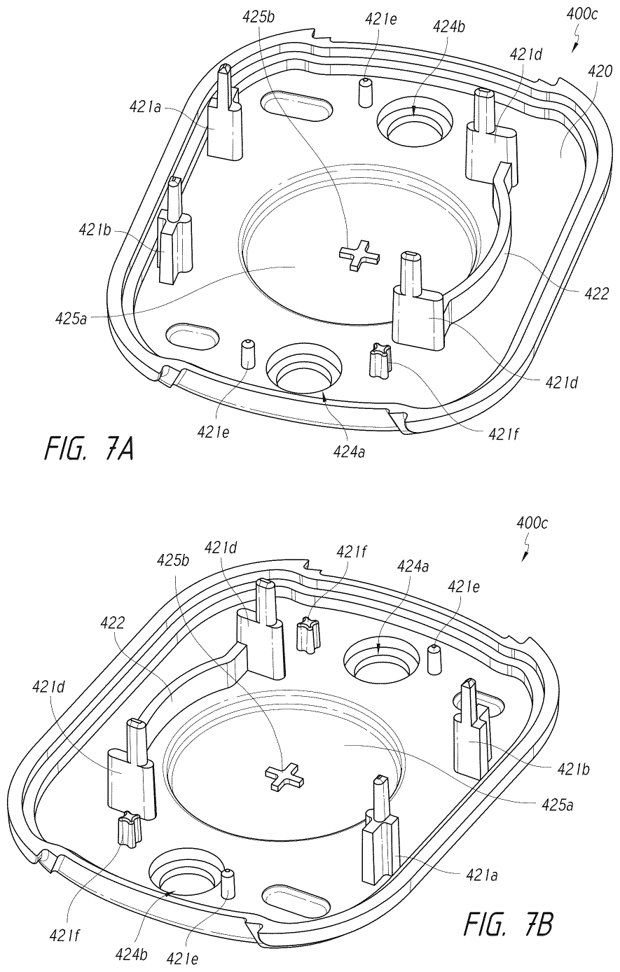

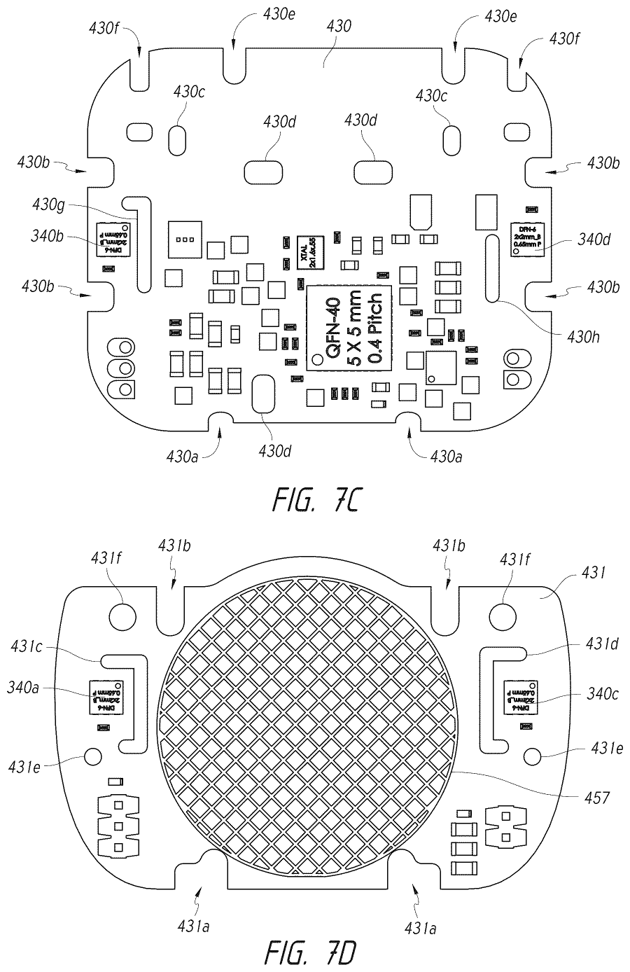

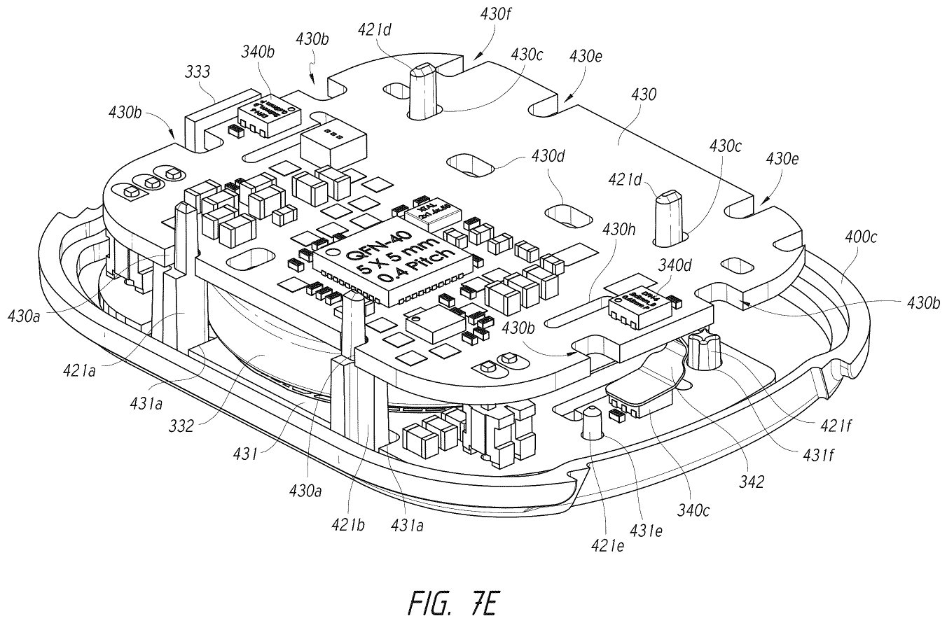

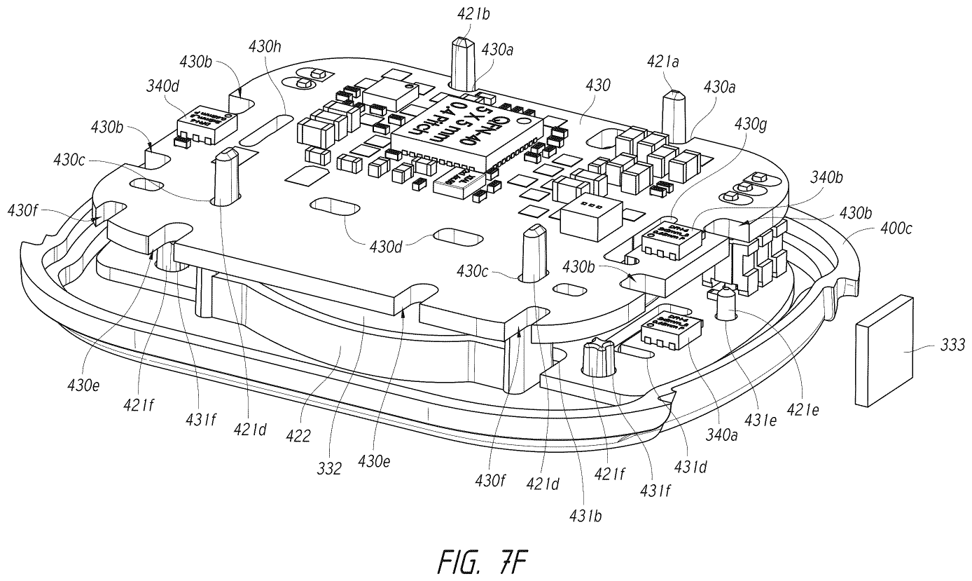

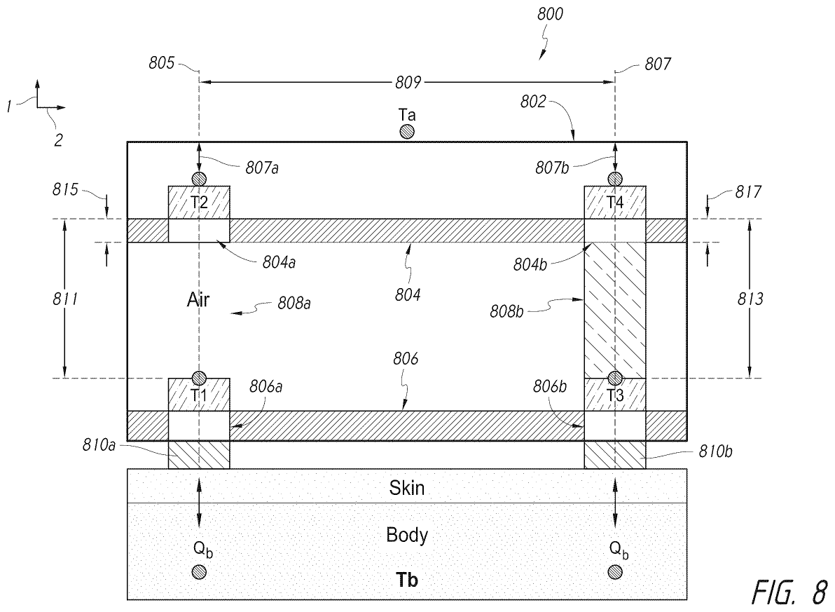

FIGS. 7A-7B illustrate another implementation of a shell 400c. Shell 400c can be utilized with shell 300a of wearable device 100. FIGS. 7C-7D illustrate top views of additional implementations of circuit boards 430, 431 that can be incorporated into wearable device 100, for example, along with shell 400c and shell 300a. FIGS. 7E-7F illustrate shell 400c assembled with circuit boards 430, 431 but without shell 300a connected thereto in order to illustrate how shell 400c and circuit boards 430, 431 can be engaged with one another as further described below. Shell 400c and circuit boards 430, 431 can be incorporated into wearable device 100 along with any of the components described elsewhere herein with respect to wearable device 100, including but not limited to battery 332, temperature sensors 340a, 340b, 340c, 340d, thermally conductive element 342, emitter 397, antenna 335, frame 334, NFC transponder 333, probes 344a, 344b, O-rings 345a, 345b, headers 336, and/or switch 325c.

Shell 400c can be similar or identical to shell 300c in some or many respects. With reference to FIGS. 7A-7B, which show top perspective views of shell 400c, shell 400c can include openings 424a, 424b, a pad 425a, and a protrusion 425b, which can be similar or identical to openings 324a, 324b, pad 325a, and protrusion 325b discussed above with reference to shell 300c. Openings 424a, 424b can allow probes 344a, 344b to extend through shell 400c, similar to openings 324a, 324b of shell 300c. Pad 325a and protrusion 325b can define, along with switch 325c, button 325 of wearable device 100. Shell 400c can include various structure(s) that can engage with portions of circuit boards 430, 431 and/or act to operably position portions of circuit boards 430, 431, similar to shell 300c and circuit boards 330, 331. For example, shell 400c can include stems 421a, 421b, 421d extending outward from surface 420 of shell 400c. Stems 421a, 421b (for example, portions thereof) can extend through and/or be received in notches 431a in circuit board 431 and notches 430a in circuit board 430 (see FIGS. 7C-7F). Stems 421 d (for example, portions thereof) can extend through and/or be received in openings 431b in circuit board 431 and openings 430c in circuit board 430 (see FIGS. 7C-7F). In the implementation illustrated in FIGS. 7A-7B, stems 421d are configured to extend through openings 430c of circuit board 430 but do not include notched gripping portions in contrast to that illustrated with respect to arms 321c, 321d of shell 300c. Shell 400c can include protrusions 421e and/or protrusions 421f which can extend outward from surface 420. Protrusions 421e can extend through openings 431e of circuit board 431 and protrusions 421f can extend through openings 431f of circuit board 431 (see FIGS. 7D-7F). In some implementations, shell 400c includes a wall 422 extending between stems 421d. Wall 422 can be similar or identical to wall 322.

With reference to FIGS. 7C-7D, circuit board 430 can include openings 430g, 430h, which can be similar or identical to openings 330g, 330h in circuit board 330. Circuit board 431 can include openings 431c, 431d, which can be similar or identical to openings 331c, 331d in circuit board 331. Circuit board 430 can include openings 430d that can be similar or identical to openings 330d in circuit board 330. Openings 430d can receive portions of walls 313a, 313b, 313c (which may also be referred as “arms” or “protrusions”) of shell 300a (see FIGS. 5G-5H). Circuit board 430 can include openings 430b (which may be referred to as “notches”) that can receive portions of walls 317a, 317b, 316a, 316b of shell 300a. Circuit board 430 can include openings 430e (which may be referred to as “notches”) and/or openings 430f (which may be referred to as “notches”) that can receive protrusions 334e and/or legs 334d of frame 334, respectively, similar or the same as how openings 330e and/or 330f of circuit board 330 can receive such protrusions 334e and/or legs 334d. FIG. 7D illustrates an electrical contact 457 that can be coupled to circuit board 431. Electrical contact 457 can be similar or identical to electrical contact 357.

### Additional Considerations and Terminology

Although this invention has been disclosed in the context of certain preferred implementations, it should be understood that certain advantages, features and aspects of the systems, devices, and methods may be realized in a variety of other implementations. Additionally, it is contemplated that various aspects and features described herein can be practiced separately, combined together, or substituted for one another, and that a variety of combination and subcombinations of the features and aspects can be made and still fall within the scope of the invention. Furthermore, the systems and devices described above need not include all of the modules and functions described in the preferred implementations.

Conditional language used herein, such as, among others, “can,” “could,” “might,” “may,” “e.g.,” and the like, unless specifically stated otherwise, or otherwise understood within the context as used, is generally intended to convey that certain features, elements, and/or steps are optional. Thus, such conditional language is not generally intended to imply that features, elements, and/or steps are in any way required or that one or more implementations necessarily include logic for deciding, with or without other input or prompting, whether these features, elements, and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain implementations require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 10 degrees, 5 degrees, 3 degrees, or 1 degree. As another example, in certain implementations, the terms “generally perpendicular” and “substantially perpendicular” refer to a value, amount, or characteristic that departs from exactly perpendicular by less than or equal to 10 degrees, 5 degrees, 3 degrees, or 1 degree.

Although certain implementations and examples have been described herein, it will be understood by those skilled in the art that many aspects of the systems and devices shown and described in the present disclosure may be differently combined and/or modified to form still further implementations or acceptable examples. All such modifications and variations are intended to be included herein within the scope of this disclosure. A wide variety of designs and approaches are possible. No feature, structure, or step disclosed herein is essential or indispensable.

Any methods disclosed herein need not be performed in the order recited. The methods disclosed herein may include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, and/or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (for example, not all described operations or events are necessary for the practice of the algorithm). Moreover, in certain implementations, operations or events can be performed concurrently, e.g., through multi-threaded processing, interrupt processing, or multiple processors or processor cores or on other parallel architectures, rather than sequentially.

Various illustrative logical blocks, modules, routines, and algorithm steps that may be described in connection with the disclosure herein can be implemented as electronic hardware (e.g., ASICs or FPGA devices), computer software that runs on general purpose computer hardware, or combinations of both. Various illustrative components, blocks, and steps may be described herein generally in terms of their functionality. Whether such functionality is implemented as specialized hardware versus software running on general-purpose hardware depends upon the particular application and design constraints imposed on the overall system. The described functionality can be implemented in varying ways for each particular application, but such implementation decisions should not be interpreted as causing a departure from the scope of the disclosure.

Moreover, various illustrative logical blocks and modules that may be described in connection with the disclosure herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can include electrical circuitry configured to process computer-executable instructions. A processor can include an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of any method, process, routine, or algorithm described in connection with the disclosure herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An example storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain implementations disclosed herein is indicated by the appended claims rather than by the foregoing description. All changes which come within the meaning and range of equivalency of the claims are to be embraced within their scope.

1.-18. (canceled)

19. A wearable device configured to secure to skin of a user, the wearable device comprising:
a first pair of temperature sensors, said first pair of temperature sensors comprising a first temperature sensor and a second temperature sensor, each of said first and second temperature sensors configured to generate one or more signals responsive to detected thermal energy, said first temperature sensor operably positioned to be closer to the user's skin than the second temperature sensor when the wearable device is secured to the user's skin;
a second pair of temperature sensors spaced from said first pair of temperature sensors, said second pair of temperature sensors comprising a third temperature sensor and a fourth temperature sensor, each of said third and fourth temperature sensors configured to generate one or more signals responsive to detected thermal energy, said third temperature sensor operably positioned to be closer to the user's skin than the fourth temperature sensor when the wearable device is secured to the user's skin; and
a thermally conductive element positioned at least partially between the third and fourth temperature sensors.

20. The wearable device of claim 19, wherein the first pair of temperature sensors are thermally insulated from one another by an air gap.

21. The wearable device of claim 20, wherein:
the wearable device further comprises a first circuit board and a second circuit board, the first and second circuit boards spaced from one another, the first circuit board positioned closer to the skin of the user than the second circuit board when the wearable device is secured to the user's skin;
said first temperature sensor is mounted to the first circuit board and the second temperature sensor is mounted to the second circuit board;
said third temperature sensor is mounted to the first circuit board and spaced from the first temperature sensor;
said fourth temperature sensor is mounted to the second circuit board and spaced from the second temperature sensor;
a distance between the first temperature sensor and the second circuit board at least partially defines said air gap; and
said thermally conductive element is positioned between the third temperature sensor and a portion of the second circuit board that is adjacent to the fourth temperature sensor.

22. The wearable device of claim 21, wherein:
said first circuit board comprises a first surface and a second surface;
said second circuit board comprises a first surface and a second surface;
said first surface of the first circuit board faces toward the second surface of the second circuit board;
said first and third temperature sensors are mounted on the first surface of the first circuit board;
said second and fourth temperature sensors are mounted on the first surface of the second circuit board; and
said thermally conductive element is positioned between the third temperature sensor and a portion of the second surface of the second circuit board that is adjacent to the fourth temperature sensor.

23. The wearable device of claim 22, wherein said second circuit board comprises at least one opening positioned between the fourth temperature sensor and a portion of said thermally conductive element, said at least one opening configured to allow thermal energy to pass from the thermally conductive element through the second circuit board and to the fourth temperature sensor.

24. The wearable device of claim 23, wherein said first and second temperature sensors are substantially aligned with one another and wherein said third and fourth temperature sensors are substantially aligned with one another.

25. The wearable device of claim 23, wherein said thermally conductive element comprises a metal strip.

26. The wearable device of claim 25, wherein said metal strip comprises copper.

27. The wearable device of claim 26, wherein the first and second circuit boards are arranged to be substantially parallel to one another.

28. The wearable device of claim 19, wherein the wearable device comprises a first portion configured to be secured to the user's skin and a second portion configured to removably secure to the first portion, and wherein one or more hardware processors, the thermally conductive element, the first pair of temperature sensors, and the second pair of temperature sensors are positioned within the second portion of the wearable device.

29. The wearable device of claim 28, wherein the first portion comprises a frame and a substrate coupled to the frame, the substrate configured to secure to the user's skin, and wherein the second portion comprises a housing.

30. The wearable device of claim 29, wherein the housing comprises a first shell and a second shell, and wherein the first and second shells are permanently secured together.

31. The wearable device of claim 19, wherein none of the first temperature sensor, second temperature sensor, third temperature sensor, and fourth temperature sensor contact the user's skin when the wearable device is secured to the user.

32. A wearable device configured to secure to skin of a user, the wearable device comprising:
a first pair of temperature sensors, said first pair of temperature sensors comprising a first temperature sensor and a second temperature sensor, said first temperature sensor operably positioned to be closer to the user's skin than the second temperature sensor when the wearable device is secured to the user's skin; and
a second pair of temperature sensors spaced from said first pair of temperature sensors, said second pair of temperature sensors comprising a third temperature sensor and a fourth temperature sensor, said third temperature sensor operably positioned to be closer to the user's skin than the fourth temperature sensor when the wearable device is secured to the user's skin.

33. The wearable device of claim 32, further comprising a thermally conductive element positioned at least partially between the third and fourth temperature sensors.

34. The wearable device of claim 33, wherein:
the first pair of temperature sensors are thermally insulated from one another by an air gap;
the wearable device further comprises a first circuit board and a second circuit board, the first and second circuit boards spaced from one another, the first circuit board positioned closer to the skin of the user than the second circuit board when the wearable device is secured to the user's skin;
said first temperature sensor is mounted to the first circuit board and the second temperature sensor is mounted to the second circuit board;
said third temperature sensor is mounted to the first circuit board and spaced from the first temperature sensor;
said fourth temperature sensor is mounted to the second circuit board and spaced from the second temperature sensor;
a distance between the first temperature sensor and the second circuit board at least partially defines said air gap; and
said thermally conductive element is positioned between the third temperature sensor and a portion of the second circuit board that is adjacent to the fourth temperature sensor.

35. The wearable device of claim 34, wherein:
said first circuit board comprises a first surface and a second surface;
said second circuit board comprises a first surface and a second surface;
said first surface of the first circuit board faces toward the second surface of the second circuit board;
said first and third temperature sensors are mounted on the first surface of the first circuit board;
said second and fourth temperature sensors are mounted on the first surface of the second circuit board; and
said thermally conductive element is positioned between the third temperature sensor and a portion of the second surface of the second circuit board that is adjacent to the fourth temperature sensor.

36. The wearable device of claim 35, wherein said second circuit board comprises at least one opening positioned between the fourth temperature sensor and a portion of said thermally conductive element, said at least one opening configured to allow thermal energy to pass from the thermally conductive element through the second circuit board and to the fourth temperature sensor.
